(19) United States
(12) Patent Application Publication
(10) Pub. No.: US 2025/0304692 A1
(43) Pub. Date: Oct. 2, 2025

(54) METHODS FOR MODULATING AN IMMUNE RESPONSE TO CANCER OR TUMOR CELLS

(71) Applicants: La Jolla Institute for Immunology, La Jolla, CA (US); The University of Liverpool, Liverpool, Merseyside (GB)

(72) Inventors: Pandurangan Vijayanand, La Jolla, CA (US); Christian Ottensmeier, Liverpool (GB); Simon Eschweiler, La Jolla, CA (US)

(73) Assignees: La Jolla Institute for Immunology, La Jolla, CA (US); The University of Liverpool, Liverpool, Merseyside (GB)

(21) Appl. No.: 18/705,402

(22) PCT Filed: Oct. 31, 2022
(86) PCT No.: PCT/US2022/048488

§ 371 Date: Apr. 26, 2024

Related U.S. Application Data
(60) Provisional application No. 63/273,760, filed on Oct. 29, 2021.

(51) Int. Cl. C07K 16/28; A61P 35/00; C07K 16/30; G01N 33/50; G01N 33/53

(52) U.S. Cl. CPC C07K 16/2815; A61P 35/00; C07K 16/2809; C07K 16/2818; C07K 16/2842; C07K 16/2851; C07K 16/2866; C07K 16/30; G01N 33/5008; G01N 33/53; C07K 2317/31; G01N 2500/10

(57) ABSTRACT

Provided herein are methods for one or more of a) modulating an immune response to a tumor cell in a patient, b) treating cancer in a cancer patient; or c) eliciting an anti-tumor response in a patient, comprising modulating the expression or activity of Junction Adhesion Molecule Like (JAML).

### CROSS-REFERENCE TO RELATED APPLICATION

This application is a U.S. National Phase Application under 35 U.S.C. § 371 of International Application No. PCT/US2022/048488, filed on Oct. 31, 2022, which claims priority under 35 U.S.C. § 119(e), and under the Paris Convention to U.S. Provisional Application No. 63/273,760, filed Oct. 29, 2021, which are hereby incorporated by reference in their entireties.

### SEQUENCE LISTING

The instant application contains a Sequence Listing which has been submitted electronically in XML file format and is hereby incorporated by reference in its entirety. Said XML copy, created on Nov. 22, 2024, is named 116639-2131_SL.xml and is 10,277 bytes in size.

### BACKGROUND OF THE DISCLOSURE

Immunotherapies targeting co-stimulatory or co-inhibitory receptors on T cells have become an important treatment option for a variety of cancer types and several novel molecules like TIM3¹, TIGIT², GITR³, VISTA⁴, LAG3⁵ or ICOS⁶ are currently being explored to evaluate their anti-tumor capacity. Crucially however, most of these targets suffer from ‘on-target/off-cell’ effects, as both effector and regulatory T cell subsets in tumor tissues can express high levels of these molecules. Applicants have previously shown that intratumoral PD-1 expressing follicular regulatory T (T_{FR}) cells are critical determinants of anti-PD-1 treatment efficacy, and that anti-PD-1 therapy can activate such suppressive cells, thus dampening treatment efficacy⁷. In line with this, it has been demonstrated that the balance of PD-1 expressing CD8⁺ T cells and T regulatory (T_{REG}) cells in the tumor microenvironment (TME) is a critical biomarker predicting anti-PD-1 treatment efficacy⁸. Also demonstrated^{9, 10-13} was the critical importance of CD8⁺ T_{RM} cells for anti-tumor immunity in multiple cancer types, and, while they have also been shown as being specific for tumor antigens¹³, so far, immunotherapies that preferentially target T_{RM} cells have not been described. These findings imply that expression levels of immunotherapy targets on different T cell subsets need to be carefully evaluated to determine which patients might benefit from a given treatment. Furthermore, while established immunotherapy drugs like anti-PD-1 or anti-CTLA-4 have shown remarkable success in some instances, only a fraction (˜20%) of patients respond to treatment¹⁴. It is well appreciated that anti-CTLA-4/anti-PD-1 combination therapy results in significantly higher overall response rates compared to monotherapy with either agent, but that combination therapy also induces more frequent and severe immune-related adverse events (irAEs) due to ‘on-target/off-tumor’ effects on T cell present in normal tissues, thus limiting its use¹⁵. Because off-cell effects and widespread immune-related toxicity severely limit both treatment efficacy and combination therapy options, there is urgent need to develop novel immunotherapy targets that exhibit a more restricted expression profile.

### SUMMARY OF THE DISCLOSURE

Junctional adhesion molecule-like protein (JAML) serves as a co-stimulatory molecule in γδ T cells with implications for tissue homeostasis and repair. While it has recently been described as a viable cancer immunotherapy target in mice, its potential to cause toxicity, specific mode of action with regard to its cellular targets, and whether it can be targeted in humans remain unknown. Here, Applicants show that JAML is readily induced by T cell receptor (TCR) engagement and revealed that this induction is mediated by cis-regulatory interactions between the CD3D and JAML gene loci in human CD8⁺ T cells, and characterized the functional consequences of JAML ligation by its endogenous ligand. When compared to other immunotherapy targets plagued by low target specificity and end-organ toxicity, it was found JAML to be mostly restricted to and highly expressed by tissue-resident memory CD8⁺ T (T_{RM}) cells in multiple cancer types. JAML expression in T_{RM} cells was associated with superior functionality, and accordingly, was also associated with improved survival outcomes in patients with head and neck squamous cell carcinoma. By delineating the key cellular targets and functional consequences of agonistic anti-JAML therapy in a murine melanoma model, Applicants uncovered its specific mode of action and the reason for its synergistic effects with anti-PD-1 and translated these findings into the disclosed therapeutic methods and compositions.

JAML was initially identified as the major co-stimulatory molecule in epithelial γδ T cells, and activation by coxsackie and adenovirus receptor (CXADR), its ligand expressed by epithelial cells, has been shown to be important for tissue homeostasis and wound repair^{16,17}. While JAML has an overall low sequence identity with the costimulatory molecule CD28 (˜11%), their intracellular signaling motifs bear substantial similarities and, upon ligation, recruit phosphatidylinositol-3-OH-kinase (PI3K), leading to cell activation, proliferation and cytokine production^{16,17}. Moreover, in mouse models, JAML has been implicated as novel cancer immunotherapy target.¹⁸

However, its role and function in tumor-infiltrating human αβ T cells, especially T_{RM} cells, remain unexplored. Applicants report herein that JAML functions as a co-stimulatory molecule in human αβ CD8⁺ T cells, and that its expression is increased by TCR signaling. Utilizing 3D chromatin interaction maps in human T cells, it is demonstrated that extensive interactions between the J4ML promoter and the neighboring CD3D) promoter region driving JAML expression in activated T cells, but not other cell compartments. Analysis of transcriptomes and protein expression data in tumor-infiltrating lymphocytes (TILs) from multiple cancer types in humans show that JAML is highly expressed by CD8⁺ T_{RM} cells and that JAML expression on CD8⁺ T_{RM} cells is associated with better survival outcomes in a large cohort of head and neck squamous cell carcinoma patients. Finally, in a murine melanoma model, it is confirmed restricted expression of JAML on CD8⁺ T cells in primary tumor tissue, but not other non-malignant organs. Crucially, Applicants report that JAML to be expressed on distinct ‘stem-like’ Tcf7^{hi}Pdcd1^{lo} and cytotoxic Pdcd1^{hi}Tcf^{lo} CD8⁺ TIL subsets, that, together with Applicants' unbiased RNA sequencing data, uncover why anti-JAML acts synergistically with anti-PD-1 therapy to augment TIL infiltration and anti-tumor immunity.

Thus, based on the disclosed observations, provided herein are methods for one or more of a) modulating an immune response to a tumor cell or cancer cell in a patient, b) treating cancer in a cancer patient; or c) eliciting an anti-tumor or anti-cancer response in a patient. The method comprises, consists of or consists essentially of modulating the expression or activity of Junction Adhesion Molecule Like (JAML). In some aspects, the modulation of JAML comprises, consists of, or consists essentially of activating the T cell by agonizing the expression or activity of JAML. In one aspect, JAML is expressed on an immune cell such as for example, a T cell. In some aspects, the T cell is selected from the group of: an activated T cell, a tissue resident memory (TRM) cell, CD8+ T cell, an αβ CD8⁺ T cell, a CD8⁺ T_{RM} cell, or a stem T cell. In some aspects, the activated T cell is specific for a tumor-associated antigen or a tumor specific antigen expressed by the tumor cell. In certain other embodiments, the antigen is optionally overexpressed or specifically expressed by the tumor cell.

In one aspect of this disclosure, the expression or activity of JAML is modulated by administering an effective amount of an agent that targets JAML expressed by the T cell. In one aspect, the agent is a JAML agonist antibody or an antigen binding fragment thereof.

In some aspects of this disclosure, the agent that targets JAML in the T cell binds to JAML and a second molecule expressed by the T cell. In some aspects, the second molecule is selected from the group of CXCR5, CXCR6, CD8, CD103, CD49A, CD69, CD3, CD28, or PD-1. In some aspects, the second molecule comprises, consists of, or consists essentially of CXCR5. In a further aspect, the agent binds to JAML in the T cell such as for example a JAML agonist antibody or an antigen binding fragment thereof and the second molecule is CXCR5.

In yet another aspect, the agent that binds to JAML comprises, consists of, or consists of an agonistic antibody targeting JAML and thus activates or augments JAML activity or expression in the T cell. In some aspects, the agent comprises, consists of, or consists essentially of a bispecific antibody (e.g., an agonist antibody or fragment thereof) that binds to JAML and a second molecule expressed by the T cell. In some aspects, the second molecule is selected from the group of CXCR5, CXCR6, CD8, CD103, CD49A, CD69, CD3, CD28, or PD-1. The bispecific antibodies of the present disclosure provide further specificity for identifying JAML expressing T cells in order to avoid undesirable off-target antibody activity. Thus, in some aspects, the bispecific antibodies only activate T cells expressing both JAML and the second molecule expressed by the T cell.

In yet another aspect of the disclosure, the agent binds to JAML and binds to a tumor or cancer antigen expressed by the tumor or cancer cell that is optionally overexpressed or specifically expressed by the tumor or cancer cell. In one aspect, one of the binding agents is a JAML agonist antibody or an antigen binding fragment thereof. In some aspects, the tumor antigen that the agent also binds comprises, consists of, or consists essentially of a tumor associated antigen or a tumor specific antigen expressed by the tumor cell. In some aspects, the tumor antigen is overexpressed by the tumor cell as compared to the expression in a normal counterpart cell. In some aspects, the tumor antigen is selected from the group of: a cancer testis antigen or a cancer embryonic antigen (CEA). In some aspects, the tumor antigen is selected from the group of. MAGE-D4B, PSMA, HER₂, HER₃, EGFR, AFP, CEA, CA-125, MUC-1, ETA, MUC-1, BAGE, GAGE-1, MAGE-A1, NY-ESO-1, Gp100, Melan-A/MART-1, Prostate-specific antigen, Mammoglobin-A, Alpha-fetoprotein, HER-2/neu, P53, K-ras, or TRP-2/INT2.

In some aspects, the tumor antigen comprises, consists of, or consists essentially of a tumor antigen that has yet to be identified. In some aspects, the agent comprises, consists of, or consists essentially of a bispecific antibody that binds to JAML and the tumor antigen.

In some aspects of this disclosure, the cancer or tumor is a cancer of at least one of the following organs: circulatory system; respiratory tract; gastrointestinal system genitourinary tract; live; bone; nervous system; reproductive system; hematologic system; oral cavity; skin and other tissues comprising connective and soft tissue, retroperitoneum and peritoneum, eye, intraocular melanoma, and adnexa, breast, head or/and neck, anal region, thyroid, parathyroid, adrenal gland colon cancer, pancreatic cancer, and other endocrine glands and related structures, and lymph nodes. The cancer may be a solid tumor or alternatively wherein the cancer is a liquid cancer, The cancer may be a primary cancer or a metastasis and/or a cancer selected from a carcinoma, a sarcoma, a myeloma, a leukemia, or lymphoma, testis cancer, brain cancer, a metastasis or recurring cancer a non-small cell lung cancer (NSCLC) and/or head and neck squamous cell cancer (HNSCC). In addition, cancer of a tissue selected from an epithelial, a head, neck, lung, prostate, colon, breast, testis, bone, lymphatic system, blood, endometrium, uterus, ovary, pancreas, esophagus, liver, skin, kidney, adrenal gland, brain. The cancer can be from the group of, a lymphoma, leukemia, breast cancer, an early-stage triple negative breast cancer, endometrial cancer, uterine, ovarian cancer, testicular cancer, lung cancer, prostate cancer, colon cancer, rectal cancer pancreatic cancer, esophageal cancer, liver cancer, melanoma, or other skin cancers, ovarian cancer, kidney cancer, adrenal gland cancer, a non-small cell lung cancer (NSCLC) and/or head and neck squamous cell cancer (HNSCC) and/or brain cancer or tumor. It can be of any stage (primary or metastatic) or a recurring tumor or cancer or neoplasia,

In some aspects, the patient is a mammal such as for example, a human patient.

In some aspects of this disclosure, the methods further comprise, consist of, or consist essentially of resecting the tumor or cancer prior to modulating the expression or activity of JAML in the T cell in the patient. In some aspects, the modulating expression or activity of JAML in a T cell is administered as a first-line, a second-line, a third-line, a fourth line or fifth line therapy.

In some aspects of this disclosure, the methods further comprise, consist of, or consist essentially of administering an effective amount of an anti-cancer agent to the patient.

In some aspects of this disclosure, the patient being treated experiences one or more of a reduced toxicity, reduction in tumor burden, longer overall survival or prolonged time to tumor progression.

In yet another aspect of this disclosure, provided herein is a method for screening for a JAML anticancer therapy comprising, consisting of, or consisting essentially of contacting a first sample containing or consisting of T cells and optionally tumor or cancer cells with an amount of the test agent that binds to JAML, and assaying for increased expression of JAML in the T cell. In some aspects, the T cell is selected from the group of: an activated T cell, a tissue resident memory (TRM) cell, CD8+ T cell, an αβ CD8⁺ T cell, a CD8⁺ T_{RM} cell, or a stem T cell. In some aspects, increased expression of JAML in the T cell is an indication that the agent is a JAML anticancer therapy. The T cells can be from patient biopsies or can be commercially obtained or cultured cells. In some aspects, the T cell in the sample is or comprises a stem T cell. Methods to determine JAML expression are known in the art and briefly described herein.

In other aspects, the test agent can be selected for sample can further comprise molecule that targets a cancer or tumor cell and the agent to be tested is specific for JAML and cancer or tumor cell. Methods to determine JAML expression are known in the art and briefly described herein. The T cell can be from patient biopsies or can be commercially obtained or cultured cells. In some aspects, the T cell is selected from the group of: an activated T cell, a tissue resident memory (TRM) cell, CD8+ T cell, an αβ CD8⁺ T cell, a CD8⁺ T_{RM} cell, or a stem T cell. In one aspect, the T cells is a stem T cell.

In yet another aspect of this disclosure, provided herein is a method for screening for a JAML anticancer therapy comprising, consisting of, or consisting essentially of contacting a first sample of T cells with an amount of the test agent that binds to JAML and a cancer or tumor antigen, and assaying for increased expression of JAML in the T cell. In some aspects, increased expression of JAML in the T cell is an indication that the agent is a JAML anticancer therapy. Methods to determine JAML expression are known in the art and briefly described herein. The sample of T cells can further comprise the cancer or tumor cell being targeted by the second agent and they can be from patient biopsies or can be commercially obtained or cultured cells. In some aspects, the T cell is selected from the group of: an activated T cell, a tissue resident memory (TRM) cell, CD8+ T cell, an αβ CD8⁺ T cell, a CD8⁺ T_{RM} cell, or a stem T cell. In some aspects, the T cell is a stem T cell. The cancer or tumor cells can be selected from the group identified above and will be selected to correspond to the test agent, e.g., a test agent comprising an anti-MAGE antibody will contain a sample comprising a cancer or tumor cell expressing MAGE. In one aspect, increased expression comprises, consists of, or consists essentially of a 2 or more, or about 3, or about 4, or about 5, or about 6, or about 7, or about 8, or about 9, or about 10, or about 11, or about 12, or about 13, or about 14, or about 15 fold increase in expression.

In yet another aspect of this disclosure, provided herein is a method of modulating JAML in a T cell in vitro or in a subject comprising, consisting of, or consisting essentially of contacting the T cell in vitro with a bispecific antibody or by administering a bispecific antibody, wherein the bispecific antibody targets and binds to JAML and a molecule expressed by a T cell. In one aspect, the bispecific antibody comprises an activating antibody or fragment thereof that binds JAML. In some aspects, the molecule expressed by the T cell is selected from CXCR5, CXCR6, CD8, CD103, CD49A, CD69, CD3, or PD-1. In some aspects, the T cell is selected from the group of: an activated T cell, a tissue resident memory (TRM) cell, CD8+ T cell, an αβ CD8⁺ T cell, a CD8⁺ T_{RM} cell, or a stem T cell. In some aspects, the T cell is a stem T cell.

In yet another aspect of this disclosure, provided herein is a method of diagnosing cancer in a subject by contacting a sample isolated from the subject with an agent that detects the presence of JAML or CXADR in the sample isolated from the subject. In one aspect, the sample comprises cells containing a T cell, e.g., a T cell selected from the group of: an activated T cell, a tissue resident memory (TRM) cell, CD8+ T cell, an αβ CD8⁺ T cell, a CD8⁺ T_{RM} cell, or a stem T cell. In some aspects, the presence of JAML or CXADR at higher or lower than baseline expression levels is diagnostic of cancer.

In yet another aspect of this disclosure, provided herein is a method of diagnosing cancer in a subject comprising, consisting of, or consisting essentially of contacting T cells isolated from the subject or tissue or cells suspected of containing cancer isolated from the subject, with an antibody or agent that recognizes and binds to JAML. If the agent binds to the cells, tissue or sample, the subject likely has cancer. In one aspect, the sample comprises cells containing a T cell, e.g., a T cell selected from the group of: an activated T cell, a tissue resident memory (TRM) cell, CD8⁺ T cell, an αβ CD8⁺ T cell, a CD8⁺ T_{RM} cell, or a stem T cell.

In yet another aspect of this disclosure, provided herein is a method of determining prognosis of a subject having cancer comprising, consisting of, or consisting essentially of measuring the density of CXADR expressing cells in a sample isolated from the subject, wherein a low density of CXADR expressing cells indicates a more positive prognosis or wherein a high density of CXADR expressing cells indicates a more negative prognosis, optionally wherein the more negative prognosis comprises a decreased probability of survival, and wherein the more positive prognosis comprises an increased probability of survival.

In yet another aspect of this disclosure, provided herein is a method of determining prognosis of a subject having cancer the method comprising, consisting of, or consisting essentially of contacting T cells isolated from the subject with an antibody or agent that recognizes and binds to JAML to determine the frequency of T cells expressing JAML in tumor cells, wherein a high frequency of JAML in T cells indicates a more positive prognosis or wherein a low frequency of JAML in T cells indicates a more negative prognosis, optionally wherein the more negative prognosis comprises a decreased probability of survival, and wherein the more positive prognosis comprises an increased probability of survival.

In yet another aspect of this disclosure, provided herein is a method of determining the responsiveness of a cancer subject to cancer therapy, the method comprising, consisting of, or consisting essentially of contacting T cells isolated from the subject with an antibody or agent that recognizes and binds to JAML to determine the frequency of JAML expressing T cells in the subject, wherein a high frequency of JAML T cells indicates an increased likelihood of responsiveness to a cancer therapy. In some aspects, the sample comprises, consists of, or consists essentially of a tumor sample. In some aspects, the cancer therapy comprises, consists of, or consists essentially of an agent that modulates the expression and/or activity of JAML in the subject. Examples of such are provided herein.

In yet another aspect of this disclosure, provided herein is a method of identifying a cancer subject that is likely to respond to a cancer therapy, comprising, consisting of, or consisting essentially of contacting a sample isolated from the subject with an agent that detects the presence of CXADR in the sample, wherein the presence of CXADR at lower than baseline expression levels indicates that the subject is likely to respond to the cancer therapy.

In some aspects of these methods, the agent that binds to JAML and/or the T cell or cancer or tumor cell can be detectably labeled or tagged. In some aspects, the detectable label or tag comprises, consists of, or consist essentially of a radioisotope, a metal, horseradish peroxidase, alkaline phosphatase, avidin or biotin.

In some aspects, baseline expression is assessed via immunohistochemistry or flow-cytometry of tissue biopsies (i.e. healthy adjacent tissue) and comprises, consists of, or consists essentially of normalized mean expression. In some aspects, expression of CXADR will be measured in tumor biopsies and compared to baseline levels, where higher than baseline expression of CXADR or JAML comprises, consists of, or consists essentially of at least about a 2 or more, or about 3, or about 4, or about 5, or about 6, or about 7, or about 8, or about 9, or about 10, or about 11, or about 12, or about 13, or about 14, or about 15 fold increase in expression relative to baseline expression and/or lower than baseline expression of CXADR or JAML is at least about a 2 or more, or about 3, or about 4, or about 5, or about 6, or about 7, or about 8, or about 9, or about 10, or about 11, or about 12, or about 13, or about 14, or about 15 fold decrease in expression relative to baseline expression.

In some aspects, the methods provided herein further comprise, consist of, or consist essentially of administering a cancer therapy to the subject. In some aspects, the cancer therapy comprises, consists of, or consists essentially of an agent that binds to JAML. In some aspects, the agent comprises, consists of, or consists essentially of an agonistic antibody targeting JAML.

In some aspects, the agent comprises, consists of, or consists essentially of a polypeptide that binds to an expression product encoded by JAML, or a polynucleotide that hybridizes to a nucleic acid sequence encoding all or a portion of JAML. In some aspects, the polypeptide comprises, consists of, or consists essentially of an antibody, an antigen binding fragment thereof, or a receptor that binds to the JAML. In some aspects, the antibody comprises, consists of, or consists essentially of an IgG, IgA, IgM, IgE or IgD, or a subclass thereof. In some aspects, the IgG comprises, consists of, or consists essentially of an IgG1, IgG2, IgG3 or IgG4. In some aspects, the antigen binding fragment comprises, consists of, or consists essentially of a Fab, Fab′, F(ab′)2, Fv, Fd, single-chain Fvs (scFv), disulfide-linked Fvs (sdFv) or VL or VH. In some aspects, the agent is contacted with the sample in conditions under which it can bind to the JAML.

In yet another aspect of this disclosure, the diagnostic, therapeutic, and/or prognostic methods provided herein further comprise, consist of or consist essentially of detection by immunohistochemistry (IHC), in-situ hybridization (ISH), ELISA, immunoprecipitation, immunofluorescence, chemiluminescence, radioactivity, X-ray, nucleic acid hybridization, protein-protein interaction, immunoprecipitation, flow cytometry, Western blotting, polymerase chain reaction, DNA transcription, Northern blotting and/or Southern blotting. In some aspects, the sample comprises, consists of, or consists essentially of cells, tissue, an organ biopsy, an epithelial tissue, a lung, respiratory or airway tissue or organ, a circulatory tissue or organ, a skin tissue, bone tissue, muscle tissue, head, neck, brain, skin, bone and/or blood sample.

### BRIEF DESCRIPTION OF THE FIGURES

FIGS. 1A-IF: JAML is enriched in tumor-infiltrating CD8⁺ T_{RM} cells of multiple cancer types. A-C, Integrated analysis of nine published single-cell RNA-seq datasets from six different cancer types visualized by UMAP depicting CD4 and CD8 T cells (A). Seurat-normalized expression of CD4 (B, left), CD8B (B, right), ITGAE (C, left) and FOAP3 (C, right) in the different clusters. D, E, Average transcript expression (shade) and percentage (size) for selected co-stimulatory (D) and co-inhibitory (E) molecules in non-T_{REG}, T_{REG}, T_{RM} and non-T_{RM} cells for integrated analysis (A-C). F, Volcano plot of JAML⁺ and JAML⁻ T_{RM} cells depicting differentially expressed transcripts (Log2 FC>0.25 and adjusted P-value <0.05) from a published single-cell RNA-seq dataset⁷.

FIGS. 2A-2E: JAML expression on T_{RM} cells is associated with patient survival. A, Whole-slide multiplexed immunohistochemistry analysis of selected markers from a treatment-naïve patient with NSCLC. B, Whole-slide multiplexed immunohistochemistry analysis depicting the percentage of JAML-expressing CD8⁺ T_{RM} (CD8⁺CD103⁺) and CD8⁺ non-T_{RM} (CD8⁺CD103⁻) cells. C, D, E Survival curves of a HNSCC cohort (n=194) stratified into T_{RM}^{h1} and T_{RM}^{lo} (C), JAML^{hi} and JAML^{lo} T_{RM} cells (D) or JAML^{hi}T_{RM}^{hi} and JAML^{low}T_{RM}^{hi} cells (E). All data are mean+/−S.E.M. Significance for comparisons was computed using two-tailed Wilcoxon matched-pairs signed rank test (B) or Mantel-Cox test (C-E), Not significant, P=0.1234; *P=0.0332; **P=0.0021; ***P=0.0002; and ****P<0.0001.

FIGS. 3A and 3B: JAML is functional in αβ T cells and is induced by TCR signaling. A, B, Flow-cytometric analysis of CD8⁺ T cells stimulated with anti-CD3+anti-CXADR, depicted is the expression of early activation markers CD69, CD25, 4-1BB and PD-1 (A) and secretion of pro-inflammatory cytokines interferon-α and tumor-necrosis factor-α. B. Depicted are the results for n=2 technical replicates (A, B). All data are representative of at least two independent experiments.

FIGS. 4A-4C: JAML expression is induced by cis-regulatory interactions between the CD3D and JAML promoters. A, JAML expression (TPM) in resting and anti-CD3 and anti-CD28-stimulated CD4⁺ and CD8⁺ T cells from donors (n=104) from a published bulk RNA-seq dataset²⁴. B, ATAC-seq, ChIP-seq tracks and HiChIP interactions for the extended JAML and CD3 gene loci in indicated cell populations, the black arrow indicates the activation-induced intronic region. C, CD3D expression (TPM) in resting and anti-CD3 and anti-CD28-stimulated CD4⁺ and CD8⁺ T cells from donors (n=104) from a published bulk RNA-seq dataset²⁴.

FIGS. 5A-5G: JAML is highly expressed by CD8⁺ TILs in a murine melanoma model. A, Representative histogram plots of in vitro stimulated CD8⁺ T cells showing the expression levels of JAML in CD8⁺ T cells treated as indicated. B,C Flow-cytometric analysis of murine CD8⁺ T cells stimulated with 0.1 μg/ml anti-CD3+ indicated concentrations of anti-JAML (B), or 0.5 μg/ml anti-CD3+ indicated concentrations of anti-JAML (C), depicted is the expression of early activation markers CD69, CD25, PD-1 and 4-1BB, depicted are the results for n=2 technical replicates. D-G, Mice were subcutaneously inoculated with B16F10-OVA cells in the right flank. Flow-cytometric analysis and representative histogram plots of the MFI of JAML in T cell populations in indicated organs at d18 after tumor inoculation (n=6 mice/group), (tumor, P<0.0001 for CD4⁺ non-T_{REG} vs CD8⁺, P<0.0001 for CD4⁺ T_{REG} vs CD8⁺; spleen, P<0.0001 for CD4⁺ non-T_{REG} vs CD8⁺, P<0.0001 for CD4⁺ T_{REG} vs CD8⁺; colon, P=0.0002 for CD4⁺ non-T_{REG} vs CD8⁺, P<0.0001 for CD4⁺ T_{REG} vs CD8⁺). Data in B-G are mean+/−S.E.M and are representative of at least 2 independent experiments. Significance for comparisons (D-G) was computed using one-way ANOVA comparing the mean of each group with the mean of the other groups followed by Tukey's test; P=0.1234; *P=0.0332; **P=0.0021; ***P=0.0002; and ****P<0.0001.

FIGS. 6A-6D JAML is expressed by distinct CD8⁺ TILs. A, B, Analysis of 10× single-cell RNA-seq data visualized by UMAP. Seurat clustering of tumor-infiltrating CD45⁺JAML⁺ cells in the B16F10-OVA model at d18 after tumor inoculation (A), Seurat-normalized expression of Pdcd1 (top) and Tcf7 (B). C, Heatmap depicting genes enriched in the identified clusters. Shown are significantly differentially expressed transcripts (Log2 FC>0.25 and adjusted P-value <0.05). D, Violin plots showing Seurat-normalized expression levels of the indicated markers in cells from cluster 0 and cluster 2.

FIGS. 7A-7E: Agonistic JAML antibody treatment impedes tumor growth. Mice were subcutaneously inoculated with B16F10-OVA cells or MC38-OVA in the right flank and treated with either isotype control antibodies, anti-PD-1 antibodies or anti-JAML antibodies at indicated time points. A, B, Tumor volume of C57BL/6J (A, n=10 mice for isotype control group and n=9 for anti-PD-1 and anti-JAML groups, P=0.0141 for isotype control vs anti-JAML and P=0.0227 for anti-PD-1 vs anti-JAML) or CD8^{-/-} (B, n=7 mice/group for isotype control and anti-JAML and n=6 mice/group for anti-PD-1) mice s.c. inoculated with B16F10-OVA cells and treated with isotype control antibodies, anti-PD-1 antibodies or anti-JAML antibodies at indicated time points. C, D, Tumor volume (C, P<0.0001 for B16F10 vs OT-I^{wt}, P=0.0014 for B16F10 vs OT-I JAML^{-/-}, P=0.033 for OT-I^{wt} vs OT-I JAML^{-/-}) (n=13 mice/group for the control group, n=8 mice/group for OT-I^{wt} and n=10 mice/group for OT-I JAML^{-/-}), and frequencies of tumor-infiltrating OT-I T cells (D, n=6 mice/group for OT-I^{wt} and n=8 mice/group for OT-I JAML^{-/-}) of mice s.c. inoculated with B16F10-OVA cells and treated with 1×10⁶ adoptively transferred wildtype OT-I T cells or JAML^{-/-} OT-I T cells at day 6 after tumor inoculation. E, Tumor volume of mice s.c. inoculated with CXADR^{+/+} or CXADR^{-/-} MC38-OVA cells and treated with either isotype control antibodies or anti-JAML antibodies at indicated time points (n=8 mice/group for CXADR^{+/+} isotype control and n=7 mice/group for CXADR^{+/+}+anti-JAML, P=0.61; n=8 mice/group for CXADR^{-/-}+isotype control and n=7 mice/group for CXADR^{-/-}+ anti-JAML, P=0.041). All data are mean+/−S.E.M and are representative of at least 2 independent experiments. Significance for comparisons was computed using two-tailed Mann-Whitney test; *P<0.05, **P<0.01, ***P<0.001 and ****P<0.0001.

FIGS. 8A-8G: Anti-JAML synergizes with anti-PD-1 therapy. Mice were subcutaneously inoculated with B16F10-OVA cells or MC38-OVA in the right flank and treated with either isotype control antibodies, anti-PD-1 antibodies or anti-JAML antibodies at indicated time points. A, Representative histogram plots depicting the gating strategy for CD4⁺ T_{REG} cells, CD4⁺ non-T_{REG} cells and CD8⁺ T cells. B, Volcano plot of isotype control vs anti-JAML (left) and isotype control vs anti-PD-1 (right) depicting differentially expressed transcripts (Log2 FC>1 and adjusted P-value <0.05). C-G, Tumor volume (C, n=7 mice/group for isotype control, n=8 mice/group for anti-JAML and CT; P=0.0225 for isotype control vs anti-JAML; P=0.0006 for isotype control vs CT), frequencies (D-G; P=0.0192 (D), P=0.0063 (E), P=0.0211 (F), P=0.0044 (G)) and representative contour plots of indicated cell populations of B16F10-OVA tumor-bearing mice treated as indicated as in (C). Data (C-G) are mean+/−S.E.M and are representative of at least 2 independent experiments. Significance for comparisons was computed using one-way ANOVA comparing the mean of each group with the mean of the control group (isotype control) followed by Dunnett's test; P=0.1234; *P=0.0332; **P=0.0021; ***P=0.0002; and ****P<0.0001.

FIGS. 9A-9B: Expression of co-stimulatory and co-inhibitory molecules in TRIG and T_{RM} cells. a, Seurat-normalized expression of LAG3, ICOS, TNFRSF9, GITR and TIGIT pertaining to (FIG. 1A). B, Flow-cytometric re-analysis depicting the percentage (A) and MFI (B) of selected co-stimulatory or co-inhibitory molecules in in non-T_{REG} (LIN⁻CD45⁺CD3⁺CD4⁺CD25⁻), T_{REG} (LIN⁻CD45⁺CD3⁺CD4⁺CD127⁻CD25⁺), T_{RM} (LIN⁻CD45⁺CD3⁺CD8⁺CD103⁺) and non-T_{RM} (LIN⁻CD45⁺CD3⁺CD8⁺CD103⁻) cells from n=10 treatment-naïve patients with NSCLC⁷.

FIGS. 10A-10E: TCR signaling induces JAML expression in human CD8⁺ T cells. A, B, Flow-cytometric analysis of anti-CD3 stimulated (A) or of anti-CD3+anti-CD28 or anti-CD3+anti-CXADR stimulated (B) CD4⁺ and CD8⁺ T cells, depicted is the expression of early activation markers CD69, CD25, 4-1BB and PD-1. Data are shown as mean of duplicates from 4 individual donors (B). C, Flow-cytometric analysis of anti-CD3+anti-CD28 or anti-CD3+anti-CXADR stimulated CD8⁺ T cells, depicted is the percentage of proliferated (Cell trace violet (CTV⁻)) cells. D, Sanger-sequencing of CD8⁺ T cells, depicted is the wildtype allele (top, CRISPR targeting irrelevant gene sequence) and the CRISPR-modified allele (bottom, CRISPR targeting depicted JAML gene sequence). Figure discloses SEQ ID NOS 6-7, 6 and 8, respectively, in order of appearance. E, PCR analysis of JAML expression from (D), depicted is the relative fold-change between the negative control guide RNA and the JAML targeting guide RNA.

FIG. 11: TCR signaling induces JAML expression in murine CD8⁺ T cells. a, ATAC-seg, ATAC-seq, ChIP-seq tracks and HiChIP interactions for the extended JAML and CD3 gene loci in indicated cell populations pertaining to (FIG. 4B).

FIGS. 12A-12C: JAML ligation activates murine CD8⁺ T cells. A-B, Flow-cytometric analysis of early activation markers, depicted are representative contour plots pertaining to (FIG. 5A). C, Flow-cytometric analysis of the frequency of CD45⁺JAML⁺ cells of B16F10-OVA tumor-bearing mice at d18 after tumor inoculation pertaining to data in (FIG. 5D-G). Data are mean+/−S.E.M and are representative of at least 2 independent experiments.

FIGS. 13A-13E: CXADR is highly expressed by cancerous cells. A, B, Representative histogram plots depicting the expression of JAML in CD8⁺ T cells (A) pertaining to (FIG. 6C, D) or CXADR in indicated tumor cells (B), grey depicts respective fluorescence minus one (FMO) control. C, D, Representative histogram plot (C) depicting the expression of CXADR in MC38-OVA cells pertaining to (FIG. 6E) and the frequency of proliferated (CTV⁻) OT-I T cells co-cultured with CXADR^{+/+} or CXADR^{-/-} MC38-OVA cells (D). E, Re-analysis of published TCGA data depicting the frequency of expression of CXADR expression in different cancer types. Data (D) are mean+/−S.E.M and are representative of at least 2 independent experiments. Significance for comparisons was computed using two-tailed Mann-Whitney test; *P<0.05, **P<0.01, ***P<0.001 and ****P<0.0001.

FIG. 14 Anti-JAML maintains a stem-like CD8⁺ T cell phenotype. Volcano plots of CD4⁺ T_{REG} cells pertaining to (FIG. 7A, B). Depicted are differentially expressed transcripts (Log2 FC>1 and adjusted P-value <0.05) in the indicated comparisons.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2% and such ranges are included. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications may be referenced by an identifying citation or by an Arabic numeral. The full citation for the publications identified by an Arabic numeral are found immediately preceding the claims. The disclosures of these publications, patents and published patent specifications are hereby incorporated by reference into the present disclosure in their entirety to more fully describe the state of the art to which this invention pertains.

### Definitions

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R.I. Freshney, ed. (1987)).

As used in the description of the invention and the appended claims, the singular forms “a,” “an” and “the” are intended to include the plural forms as well, unless the context clearly indicates otherwise.

As used herein, the term “comprising” is intended to mean that the compositions and methods include the recited elements, but do not exclude others. As used herein, the transitional phrase consisting essentially of (and grammatical variants) is to be interpreted as encompassing the recited materials or steps and those that do not materially affect the basic and novel characteristic(s) of the recited embodiment. Thus, the term “consisting essentially of” as used herein should not be interpreted as equivalent to “comprising”. “Consisting of” shall mean excluding more than trace elements of other ingredients and substantial method steps for administering the compositions disclosed herein. Aspects defined by each of these transition terms are within the scope of the present disclosure.

The term “about” as used herein when referring to a measurable value such as an amount or concentration and the like, is meant to encompass variations of 20%, 10%, 5%, 1%, 0.5%, or even 0.1% of the specified amount.

As used herein, the terms “increased”, “decreased”, “high”, “low” or any grammatical variation thereof refer to a variation of about 90%, 80%, 50%, 20%, 10%, 5%, 1%, 0.5%, or even 0.1% of the reference composition, polypeptide, protein, etc.

The phrase “lacks expression of” a protein/polypeptide refers to that (i) the protein/polypeptide is note encoded or present, and/or (2) the protein/polypeptide is present at a low level compared to a control (for example, a non-cancer cell or tissue).

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

An equivalent of a polynucleotide (referred to herein as the reference) shares at least 50% (or at least 60%, or at least 70%, or at least 80%, or at least 90%) identity to the reference, and encodes the same polypeptide as the one encoded by the reference, or encodes an equivalent of the polypeptide encoded by the reference.

To arrive at a position or a consecutive segment of a test sequence equivalent to (or corresponding to) an/a amino acid/nucleotide residue or a consecutive segment of a reference sequence, a sequence alignment is performed between the test and reference sequences. The positions or segments aligned to each other are determined as equivalents.

The term “analogue” refers to an equivalent having one or more modified amino acids and one or more amino acids replaced with another amino acid. Such modification may include but is not limited to conjugation with a molecule (for example, a small molecule, a cytotoxic molecule, a linker, a pH-sensitive linker, and/or a thiol linker), sialylation, polysialylation, O-glycosylation, N-glycosylation, myristoylation, palmitoylation, isoprenylation or prenylation, glipyatyon, lipoylation, phosphopantetheinylation, ethanolamine phosphoglycerol attachment, diphthamide formation, hypusine formation, acylation, acetylation, formylation, alkylation, methylation, amidation, citrullination, deamidation, eliminylation, ISGylation, SUMOylation, ubiquitination, neddylation, pupylation, biotinylation, carbamylation, oxidation, pegylation, glycation, carbamylation, carbonylation, spontaneous isopeptide bond formation, butyrylation, gamma-carboxylation, malonylation, hydroxylation, iodination, nucleotide addition, phosphate ester (O-linked) or phosphoramidate (N-linked) formation, phosphorylation, adenylylation, uridylylation, propionylation, pyroglutamate formation, S-glutathionylation, S-nitrosylation, S-sulfenylation, S-sulfinylation, S-sulfonylation, succinylation, and/or sulfation. The term “albumin equivalent,” comprises, or consists essentially of, or yet further consists of, polypeptides which can be expressed at a reasonable quantity and which still retains or improves on certain albumin properties, including the binding of the albumin fragment to an FcRn receptor, as is known in the art or described herein.

The term “affinity tag” refers to a polypeptide that may be included within a fusion protein to allow detection of the fusion protein and/or purification of the fusion protein from the cellular milieu using a ligand that is able to bind to, i.e., has affinity for, the affinity tag. The ligand may be, but is not limited to, an antibody, a resin, or a complementary polypeptide. An affinity tag may comprise a small peptide, commonly a peptide of approximately 4 to 16 amino acids in length, or it may comprise a larger polypeptide. Commonly used affinity tags include polyarginine, FLAG, V5, polyhistidine, c-Myc, Strep II, maltose binding protein (MBP), N-utilization substance protein A (NusA), thioredoxin (Trx), and glutathione S-transferase (GST), among others (for examples, see GST Gene Fusion System Handbook-Sigma-Aldrich). In an embodiment the affinity tag is a polyhistidine tag, for example a His6 tag (SEQ ID NO: 1). The inclusion of an affinity tag in a fusion protein allows the fusion protein to be purified from the cellular milieu by affinity purification, using an affinity medium that is able to tightly and specifically bind the affinity tag. The affinity medium may comprise, for example, a metal-charged resin or a ligand covalently linked to a stationary phase (matrix) such as agarose or metal beads. For example, polyhistidine tagged fusion proteins (also referred to as His tagged fusion proteins) can be recovered by immobilized metal ion chromatography using Ni²⁺ or Co²⁺ loaded resins, anti-FLAG affinity gels may be used to capture FLAG tagged fusion proteins, and glutathione cross-linked to a solid support such as agarose may be used to capture GST tagged fusion proteins.

As used herein the terms “purification”, “purifying”, or “separating” refer to the process of isolating one or more polypeptides from a complex mixture, such as a cell lysate or a mixture of polypeptides. The purification, separation, or isolation need not be complete, i.e., some components of the complex mixture may remain with the one or more polypeptides after the purification process. However, the product of purification should be enriched for the one or more polypeptides relative to the complex mixture before purification and a significant portion of the other components initially present within the complex mixture should be removed by the purification process.

The term “cell” as used herein may refer to either a prokaryotic or eukaryotic cell, optionally obtained from a subject or a commercially available source.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human, e.g., HEK293 cells, Chinese Hamster Ovary (CHO) cells and 293T cells.

“Prokaryotic cells” that usually lack a nucleus or any other membrane-bound organelles and are divided into two domains, bacteria and archaea. In addition to chromosomal DNA, these cells can also contain genetic information in a circular loop called an episome. Bacterial cells are very small, roughly the size of an animal mitochondrion (about 1-2 m in diameter and 10 m long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality (for example, having a similar functional activity). It should be understood, without being explicitly stated that when referring to an equivalent or biological equivalent to a reference polypeptide, protein, or polynucleotide, that an equivalent or biological equivalent has the recited structural relationship to the reference polypeptide, protein, or polynucleotide and equivalent or substantially equivalent biological activity. For example, non-limiting examples of equivalent polypeptides, proteins, or polynucleotides include a polypeptide, protein or polynucleotide having at least 60%, or alternatively at least 65%, or alternatively at least 70%, or alternatively at least 75%, or alternatively 80%, or alternatively at least 85%, or alternatively at least 90%, or alternatively at least 95% identity thereto or for polypeptide, polynucleotide or protein sequences across the length of the reference polynucleotide. Alternatively, an equivalent polypeptide is one that is encoded by a polynucleotide or its complement that hybridizes under conditions of high stringency to a polynucleotide encoding such reference polypeptide sequences and that have substantially equivalent or equivalent biological activity. Conditions of high stringency are described herein and incorporated herein by reference. Alternatively, an equivalent thereof is a polypeptide encoded by a polynucleotide or a complement thereto, having at least 70%, or alternatively at least 75%, or alternatively 80%, or alternatively at least 85%, or alternatively at least 90%, or alternatively at least 95% identity, or at least 97% sequence identity across the length of the reference polynucleotide to the reference polynucleotide, e.g., the wild-type polynucleotide. Such equivalent polypeptides have the same biological activity as the reference polynucleotide.

Non-limiting examples of equivalent polypeptides, include a polynucleotide having at least 60%, or alternatively at least 65%, or alternatively at least 70%, or alternatively at least 75%, or alternatively 80%, or alternatively at least 85%, or alternatively at least 90%, or alternatively at least 95%, or alternatively at least 97%, identity to a reference polynucleotide. An equivalent also intends a polynucleotide or its complement that hybridizes under conditions of high stringency to a reference polynucleotide. Such equivalent polypeptides have the same biological activity as the reference polynucleotide.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences across the length of the reference polynucleotide. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter-none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity can be determined by incorporating them into clustalW (available at the web address:genome.jp/tools/clustalw/, last accessed on Jan. 13, 2017).

“Homology” or “identity” or “similarity” refers to sequence similarity between two peptides or between two nucleic acid molecules. Homology can be determined by comparing a position in each sequence that may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

As used herein, the term “at least 90% identical” refers to an identity of two compared sequences (polynucleotides or polypeptides) of about 90% to about 100%. It also include an identity of at least at least 91%, at least 92%, at least 93%, at least 94%, at least 95%, at least 96%, at least 97%, at least 98%, at least 99%, about 91% to about 100%, about 92% to about 100%, about 93% to about 100%, about 94% to about 100%, about 95% to about 100%, about 96% to about 100%, about 97% to about 100%, about 98% to about 100%, or about 99% to about 100%.

“Homology” or “identity” or “similarity” can also refer to two nucleic acid molecules that hybridize under stringent conditions.

As used herein, the terms “retain” “similar” and “same” are used interchangeably while describing a function, an activity or an functional activity of a polynucleotide, a protein and/or a peptide, referring to a functional activity of at least about 20% (including but not limited to: at least about 30%, at least about 40%, at least about 50%, at least about 60%, at least about 70%, at least about 80%, at least about 90%, at least about 95%, at least about 97%, or about 100%) of the activity of the reference protein, polynucleotide and/or peptide.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. In one aspect, an equivalent polynucleotide is one that hybridizes under stringent conditions to a reference polynucleotide or its complement. In another aspect, an equivalent polypeptide is a polypeptide that is encoded by a polynucleotide is one that hybridizes under stringent conditions to a reference polynucleotide or its complement.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “isolated” or a grammatical variation thereof as used herein refers to molecules or biologicals or cellular materials being substantially free from other materials.

As used herein, the term “functional” may be used to modify any molecule, biological, or cellular material to intend that it accomplishes a particular, specified effect.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, complementary DNA (cDNA), DNA-RNA hybrids, or a polymer comprising purine and pyrimidine bases or other natural, chemically or biochemically modified, non-natural, or derivatized nucleotide bases. In certain embodiments, the polynucleotide comprises and/or encodes a messenger RNA (mRNA), a short hairpin RNA, and/or small hairpin RNA. In one embodiment, the polynucleotide is or encodes an mRNA. In certain embodiments, the polynucleotide is a double-strand (ds) DNA, such as an engineered ds DNA or a ds cDNA synthesized from a single-stranded RNA.

As used herein, the terms “engineered” “synthetic” “recombinant” and “non-naturally occurring” are interchangeable and indicate intentional human manipulation, for example, a modification from its naturally occurring form, and/or a sequence optimization.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, a consecutive amino acid sequence refers to a sequence having at least two amino acids. However, it is noted that a consecutive amino acid sequence of a first part and a second part does not limit the amino acid sequence to have the first part directly conjugated to the second part. It is also possible that the first part is linked to the second part via a third part, such as a link, thus forming one consecutive amino acid sequence.

A polynucleotide disclosed herein can be delivered to a cell or tissue using a gene delivery vehicle. “Gene delivery,” “gene transfer” “mRNA-based delivery”, “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes, including for example protamine complexes, lipid nanoparticles, polymeric nanoparticles, lipid-polymer hybrid nanoparticles, and inorganic nanoparticles, or combinations thereof) as well as techniques facilitating the delivery of “naked” polynucleotides (such as electroporation, “gene gun” delivery and various other techniques used for the introduction of polynucleotides). The introduced polynucleotide can be unmodified or can comprise one or more modifications; for example, a modified mRNA may comprise ARCA capping; enzymatic polyadenylation to add a tail of 100-250 adenosine residues (SEQ ID NO: 2); and substitution of one or both of cytidine with 5-methylcytidine and/or uridine with pseudouridine. The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearized by using restriction enzymes, and then DNA ligase can add a sequence or gene of interest within the linear molecule by the use of cohesive ends. Yeast expression vectors, such as YACs, YIps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

A “viral vector” is defined as a recombinantly produced virus or viral particle that comprises a polynucleotide to be delivered into a host cell, either in vivo, ex vivo or in vitro.

Examples of viral vectors include retroviral vectors, adenovirus vectors, adeno-associated virus vectors, herpes simplex virus vectors, alphavirus vectors and the like.

As used herein, the term “animal” refers to living multi-cellular vertebrate organisms, a category that includes, for example, mammals and birds. The term “mammal” includes both human and non-human mammals.

The terms “subject,” “host,” “individual,” and “patient” are as used interchangeably herein. A “subject” of diagnosis or treatment is a cell or an animal such as a mammal, or a human. A subject is not limited to a specific species and includes non-human animals subject to diagnosis or treatment and are those subject to infections or animal models, for example, simians, murines, such as, rats, mice, chinchilla, canine, such as dogs, leporids, such as rabbits, livestock, sport animals, and pets. Human patients are included within the term as well.

In one aspect, a “subject” or “patient” to whom the therapies such as for example a combination of anti-JMAL therapy and immune checkpoint inhibitor is administered is preferably a mammal such as a non-primate (e.g., cow, pig, horse, cat, dog, rat, etc.) or a primate (e.g., monkey or human). The subject or patient can be a human, such as an adult patient or a pediatric patient.

An “effective amount” or “efficacious amount” refers to the amount of an agent, or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

As used herein, a biological sample, or a sample, can be obtained from a subject, cell line or cultured cell or tissue. Exemplary samples include, but are not limited to, cell sample, tissue sample, tumor biopsy, liquid samples such as blood and other liquid samples of biological origin (including, but not limited to, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some instances, the sample is a tumor/cancer biopsy.

A “solid tumor” is an abnormal mass of tissue that usually does not contain cysts or liquid areas. Solid tumors can be benign or malignant. Different types of solid tumors are named for the type of cells that form them. Examples of solid tumors include sarcomas, carcinomas, and lymphomas. The solid tumor can be localized or metastatic.

In certain embodiments, the terms “disease” “disorder” and “condition” are used interchangeably herein, referring to a cancer, a status of being diagnosed with a cancer, or a status of being suspect of having a cancer.

As used herein, a “cancer” is a disease state characterized by the presence in a subject of cells demonstrating abnormal uncontrolled replication and may be used interchangeably with the term “tumor.” In some embodiments, the cancer is a leukemia or a lymphoma. “Cell associated with the cancer” refers to those subject cells that demonstrate abnormal uncontrolled replication. In certain embodiments, the cancer is acute myeloid leukemia or acute lymphoblastic leukemia. As used herein a “leukemia” is a cancer of the blood or bone marrow characterized by an abnormal increase of immature white blood cells. The specific condition of acute myeloid leukemia (AML)—also referred to as acute myelogenous leukemia or acute myeloblastic leukemia—is a cancer of the myeloid origin blood cells, characterized by the rapid growth of abnormal myeloid cells that accumulate in the bone marrow and interfere with the production of normal blood cells. The specific condition of acute lymphoblastic leukemia (ALL)—also referred to as acute lymphocytic leukemia or acute lymphoid leukemia—is a cancer of the white blood cells, characterized by the overproduction and accumulation of malignant, immature leukocytes (lymphoblasts) resulting a lack of normal, healthy blood cells. As used herein a “lymphoma” is a cancer of the blood characterized by the development of blood cell tumors and symptoms of enlarged lymph nodes, fever, drenching sweats, unintended weight loss, itching, and constantly feeling tired.

As used herein, a “cancer” is a disease state characterized by the presence in a subject of cells demonstrating abnormal uncontrolled replication and may be used interchangeably with the term “tumor.” In some embodiments, the cancer is a leukemia or a lymphoma. “Cell associated with the cancer” refers to those subject cells that demonstrate abnormal uncontrolled replication. In certain embodiments, the cancer is acute myeloid leukemia or acute lymphoblastic leukemia. As used herein a “leukemia” is a cancer of the blood or bone marrow characterized by an abnormal increase of immature white blood cells. The specific condition of acute myeloid leukemia (AML)—also referred to as acute myelogenous leukemia or acute myeloblastic leukemia—is a cancer of the myeloid origin blood cells, characterized by the rapid growth of abnormal myeloid cells that accumulate in the bone marrow and interfere with the production of normal blood cells. The specific condition of acute lymphoblastic leukemia (ALL)—also referred to as acute lymphocytic leukemia or acute lymphoid leukemia—is a cancer of the white blood cells, characterized by the overproduction and accumulation of malignant, immature leukocytes (lymphoblasts) resulting a lack of normal, healthy blood cells. As used herein a “lymphoma” is a cancer of the blood characterized by the development of blood cell tumors and symptoms of enlarged lymph nodes, fever, drenching sweats, unintended weight loss, itching, and constantly feeling tired.

“Cancer”, which is also referred to herein as “tumor”, is a known medically as an uncontrolled division of abnormal cells in a part of the body, benign or malignant. In one embodiment, cancer refers to a malignant neoplasm, a broad group of diseases involving unregulated cell division and growth, and invasion to nearby parts of the body. Non-limiting examples of cancers include carcinomas, sarcomas, leukemia and lymphoma, e.g., colon cancer, colorectal cancer, rectal cancer, gastric cancer, esophageal cancer, head and neck cancer, breast cancer, brain cancer, lung cancer, stomach cancer, liver cancer, gall bladder cancer, or pancreatic cancer. In one embodiment, the term “cancer” refers to a solid tumor, which is an abnormal mass of tissue that usually does not contain cysts or liquid areas, including but not limited to, sarcomas, carcinomas, and certain lymphomas (such as Non-Hodgkin's lymphoma). In another embodiment, the term “cancer” refers to a liquid cancer, which is a cancer presenting in body fluids (such as, the blood and bone marrow), for example, leukemias (cancers of the blood) and certain lymphomas.

Additionally or alternatively, a cancer may refer to a local cancer (which is an invasive malignant cancer confined entirely to the organ or tissue where the cancer began), a metastatic cancer (referring to a cancer that spreads from its site of origin to another part of the body), a non-metastatic cancer, a primary cancer (a term used describing an initial cancer a subject experiences), a secondary cancer (referring to a metastasis from primary cancer or second cancer unrelated to the original cancer), an advanced cancer, an unresectable cancer, or a recurrent cancer. As used herein, an advanced cancer refers to a cancer that had progressed after receiving one or more of: the first line therapy, the second line therapy, or the third line therapy.

As used herein, the term “extracellular matrix” (ECM) is a three-dimensional network of extracellular macromolecules, such as collagen, enzymes, and glycoproteins, that provide structural and biochemical support to surrounding cells. It is an essential component of the tumor microenvironment. Cancer development and progression are associated with increased ECM deposition and crosslink, while the chemical and physical signals elicited from ECM are necessary for cancer cell proliferation and invasion. In one embodiment, the ECM of a cancer comprises a peri-cancerous cell or tissue.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6 phoshpate, dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S, ⁸⁹Zr or ¹²⁵I.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP (SEQ ID NO: 9)), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, “immunophenotyping” refers to the analysis of heterogeneous populations of cells for the purpose of identifying the presence and proportions of the various populations in the sample. Antibodies are used to identify cells by detecting specific antigens (termed markers) expressed by these cells. In an aspect, the cell samples are characterized by immunophenotyping using techniques such as flow cytometry. In alternative aspects, characterizations of the various cell types, (such as T cells, B cells and their subsets) present in a cell sample may be carried out using any suitable methodology such as reverse transcriptase polymerase chain reaction (RT-PCR) or immunocytochemistry (IHC).

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment”. Typically, a patient is given a subsequent chemotherapy regimen because the patient did not show a positive clinical or sub-clinical response to the first line therapy or the first line therapy has stopped.

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg), Tissue-resident memory T cells (T_{RM} cells), stem T cells and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC #CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PERO117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4;11 (ATCC CRL-1873), CCRF-CEM (ATCC CRM-CCL-119); and cutaneous T-cell lymphoma lines, e.g., HuT78 (ATCC CRM-TIB-161), MJ[G11](ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

“Tissue resident memory cell” or “TRM” cells refer to a subset of long-lived memory T cells that occupy epithelial and mucosal tissues.

“Stem T cells” refer to a subset of lymphocytes with the stem-like ability to self-renew and the multipotent capacity to reconstitute various memory and effector cell subsets. The antibodies disclosed herein may target and bind to TRMs or stem T cells in order to enhance a specific T cell population and increasing the population of effector or memory T cells against tumor and cancer cells. Stem T cells are capable of self-renewal and may prevent T cell exhaustion.

“Frequency” of cells expressing any one particular molecule, biomarker, or antigen refers to the likelihood of or ratio of cells expressing the molecule, biomarker, or antigen compared to a population of T cells at large.

“Density” of cells expressing any one particular molecule biomarker, or antigen refers to the amount or mass of cells expressing the molecule, biomarker, or antigen in a given sample per volume of sample.

As used herein, the term “engineered T-cell receptor” refers to a molecule comprising the elements of (a) an extracellular antigen binding domain, (b) a transmembrane domain, and (c) an intracellular signaling domain. In some aspect, an engineered T-cell receptor is a genetically modified TCR, a modified TCR, a recombinant TCR, a transgenic TCR, a partial TCR, a chimeric fusion protein, a CAR, a first generation CAR, a second generation CAR, a third generation CAR, or a fourth generation TRUCK. In some aspect, the engineered T-cell receptor comprises an antibody or a fragment of an antibody. In particular aspects, the engineered T-cell receptor is a genetically modified TCR or a CAR.

As used herein, the term “receptor” or “T-cell receptor” or “TCR” refers to a cell surface molecule found on T-cells that functions to recognize and bind antigens presented by antigen presenting molecules. Generally, a TCR is a heterodimer of an alpha chain (TRA) and a beta chain (TRB). Some TCRs are comprised of alternative gamma (TRG) and delta (TRD) chains. T-cells expressing this version of a TCR are known as γδ T-cells. TCRs are part of the immunoglobulin superfamily. Accordingly, like an antibody, the TCR comprises three hypervariable CDR regions per chain. There is also an additional area of hypervariability on the beta-chain (HV4). The TCR heterodimer is generally present in an octomeric complex that further comprises three dimeric signaling modules CD37γ/ε, CD3δ/ε, and CD247ζ/ζ or ζ/η. Non-limiting exemplary amino acid sequence of the human TCR-alpha chain:

(SEQ ID NO: 3)
METLLGVSLVILWLQLARVNSQQGEEDPQALSIQEGENATMNCSY
KTSINNLQWYRQNSGRGLVHLILIRSNEREKHSGRLRVTLDTSKK
SSSLLITASRAADTASYFCAPVLSGGGADGLTFGKGTHLIIQPYI
QNPDPAVYQLRDSKSSDKSVCLFTDFDSQTNVSQSKDSDVYITDK
TVLDMRSMDFKSNSAVAWSNKSDFACANAFNNSIIPEDTFFPSPE
SSCDVKLVEKSFETDTNLNFQNLSVIGFRILLLKVAGFNLLMTLR
LWSS.

Non-limiting exemplary amino acid sequence of the human TCR-beta chain:

(SEQ ID NO: 4)
DSAVYLCASSLLRVYEQYFGPGTRLTVTEDLKNVFPPEVAVFEPP
EAEISHTQKATLVCLATGFYPDHVELSWWVNGKEVHSGVSTDPQP
LKEQP.

The term “modified TCR” refers to a TCR that has been genetically engineered, and/or a transgenic TCR, and/or a recombinant TCR. Non-limiting examples of modified TCRs include single-chain VαVβ TCRs (scTv), full-length TCRs produced through use of a T cell display system, and TCRs wherein the CDR regions have been engineered to recognize a specific antigen, peptide, fragment, and/or MHC molecule. Methods of developing and engineering modified TCRs are known in the art. For example, see Stone, J. D. et al. Methods in Enzymology 503: 189-222 (2012), PCT Application WO2014018863 A1.

As used herein, the terms “antibody,” “antibodies” and “immunoglobulin” includes whole antibodies and any antigen binding fragment or a single chain thereof. Thus the term “antibody” includes any protein or peptide containing molecule that comprises at least a portion of an immunoglobulin molecule. The terms “antibody,” “antibodies” and “immunoglobulin” also include immunoglobulins of any isotype, fragments of antibodies which retain specific binding to antigen, including, but not limited to, Fab, Fab′, F(ab)₂, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues. The term “anti-” when used before a protein name, anti-JAML for example, refers to a monoclonal or polyclonal antibody that binds and/or has an affinity to a particular protein.

The antibodies can be polyclonal, monoclonal, multispecific (e.g., bispecific antibodies), and antibody fragments, so long as they exhibit the desired biological activity. Antibodies can be isolated from any suitable biological source, e.g., murine, rat, sheep and canine.

The term “bispecific antibody” refers to an antibody that can simultaneously bind to two different receptors, epitopes or antigens. The bispecific antibodies of the instant disclosure may target and bind antigens on the same cells or different cells. In some aspects, the bispecific antibodies bind to JAML and a second molecule on the T cell. JAML may be expressed on a T cell. In some aspects, the second molecule is expressed on the same T cell. In one aspect, the bispecific antibodies of the claimed disclosure increase target specificity for JAML expressing T cells, while limiting undesirable off-target activity. In some aspects, the bispecific bind and modulate the expression or activity of JAML in or on the T cell or the JAML expressing T cells.

In some other aspects as described herein, the bispecific antibodies bind to JAML and a tumor or cancer antigen expressed by a tumor or cancer cell, including but not limited to tumor associated antigens or tumor specific antigens. The bispecific antibody may simultaneously bind and activate the JAML expressing T cell, while also binding a tumor or cancer antigen. Thus, the activated T cell is able to target the tumor or cancer cells expressing the antigen. In some aspects the antigen is overexpressed or specifically expressed by the tumor or cancer cell. Therefore, the bispecific antibodies of the present disclosure can be configured to bind to overexpressed or specifically expressed tumor or cancer antigens, including tumor associated or tumor specific antigens, that are identifiable markers of the tumor or cancer cell, rather than undesirably binding to off-target cells and antigens.

As used herein, “monoclonal antibody” refers to an antibody obtained from a substantially homogeneous antibody population. Monoclonal antibodies are highly specific, as each monoclonal antibody is directed against a single determinant on the antigen. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

Monoclonal antibodies may be generated using hybridoma techniques or recombinant DNA methods known in the art. A hybridoma is a cell that is produced in the laboratory from the fusion of an antibody-producing lymphocyte and a non-antibody producing cancer cell, usually a myeloma or lymphoma. A hybridoma proliferates and produces a continuous sample of a specific monoclonal antibody. Alternative techniques for generating or selecting antibodies include in vitro exposure of lymphocytes to antigens of interest, and screening of antibody display libraries in cells, phage, or similar systems.

The term “human antibody” as used herein, is intended to include antibodies having variable and constant regions derived from human germline immunoglobulin sequences. The human antibodies disclosed herein may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

A “human monoclonal antibody” refers to antibodies displaying a single binding specificity which have variable and constant regions derived from human germline immunoglobulin sequences. The term also intends recombinant human antibodies. Methods to making these antibodies are described herein.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. Methods to making these antibodies are described herein.

As used herein, chimeric antibodies are antibodies whose light and heavy chain genes have been constructed, typically by genetic engineering, from antibody variable and constant region genes belonging to different species.

As used herein, the term “humanized antibody” or “humanized immunoglobulin” refers to a human/non-human chimeric antibody that contains a minimal sequence derived from non-human immunoglobulin. For the most part, humanized antibodies are human immunoglobulins (recipient antibody) in which residues from a variable region of the recipient are replaced by residues from a variable region of a non-human species (donor antibody) such as mouse, rat, rabbit, or non-human primate having the desired specificity, affinity and capacity. Humanized antibodies may comprise residues that are not found in the recipient antibody or in the donor antibody. The humanized antibody can optionally also comprise at least a portion of an immunoglobulin constant region (Fc), typically that of a human immunoglobulin, a non-human antibody containing one or more amino acids in a framework region, a constant region or a CDR, that have been substituted with a correspondingly positioned amino acid from a human antibody. In general, humanized antibodies are expected to produce a reduced immune response in a human host, as compared to a non-humanized version of the same antibody. The humanized antibodies may have conservative amino acid substitutions which have substantially no effect on antigen binding or other antibody functions. Conservative substitutions groupings include: glycine-alanine, valine-leucine-isoleucine, phenylalanine-tyrosine, lysine-arginine, alanine-valine, serine-threonine and asparagine-glutamine. The antibodies of the present invention may bind to an antigen or molecule. In some aspects, the antigens or molecules are expressed in a T cell, tumor cell, or tissue of a subject.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope.

As used herein, the term “antibody derivative”, comprises a full-length antibody or a fragment of an antibody, wherein one or more of the amino acids are chemically modified by alkylation, pegylation, acylation, ester formation or amide formation or the like, e.g., for linking the antibody to a second molecule. This includes, but is not limited to, pegylated antibodies, cysteine-pegylated antibodies, and variants thereof.

An agonistic anti-junctional adhesion molecule-like protein (JAML) antibody (anti-JAML antibody) intends an antibody, antigen binding fragment, derivative or other modification as described herein and known in the art that recognizes and binds the JAML protein. The protein sequence of JAML is publicly available at https://www.uniprot.org/uniprot/Q86YT9 (accessed on Oct. 28, 2021) and reproduced below:

```
(SEQ ID NO: 5)
                10 20 30 40 50
MFCPLKLILL PVLLDYSLGL NDLNVSPPEL TVHVGDSALM GCVFQSTEDK
                60 70 80 90 100
CIFKIDWTLS PGEHAKDEYV LYYYSNLSVP IGRFQNRVHL MGDILCNDGS
              110 120 130 140 150
LLLQDVQEAD QGTYICEIRL KGESQVFKKA VVLHVLPEEP KELMVHVGGL
              160 170 180 190 200
IQMGCVFQST EVKHVTKVEW IFSGRRAKEE IVFRYYHKLR MSVEYSQSWG
              210 220 230 240 250
HFQNRVNLVG DIFRNDGSIM LQGVRESDGG NYTCSIHLGN LVFKKTIVLH
              260 270 280 290 300
VSPEEPRTLV TPAALRPLVL GGNQLVIIVG IVCATILLLP VLILIVKKTC
              310 320 330 340 350
GNKSSVNSTV LVKNIKKINP EIKEKPCHFE RCEGEKHIYS PIIVREVIEE
              360 370 380 390
EEPSEKSEAT YMTMHPVWPS LRSDRNNSLE KKSGGGMPKT QQAF
```

Polyclonal and monoclonal antibodies that bind JAML are known in the art and commercially available, see Creative Biolabs, Sino Biological, Invitrogen (monoclonal and polyclonal).

“Antigen” broadly refers to a molecule or molecular structure that can bind to a specific antibody or T-cell receptor. In some aspects, the antigen may be expressed by a cell. In other aspects, the antigen may be a tumor cell antigen. A “tumor cell antigen” or “tumor antigen” refers to an antigen or antigenic substance produced by tumor cells or cancer cells. Tumor associated antigens (TAAs) are antigens that are present on tumor cells and also normal cells. In some aspects, the TAA may be overexpressed or underexpressed by the tumor cell relative to normal cells. Tumor specific antigens (TSAs) are antigens that may only be expressed by tumor cells and may not be expressed on any other cells.⁴¹ Tumor cell antigens of the instant disclosure include both known and yet to be identified tumor cell antigens.

Cancer-testis antigens (CTAs) form a family of antigens that are encoded by 276 genes, comprising more than 70 gene families, whose expression is typically restricted to testicular germ cells and placenta trophoblasts with no or low expression in normal adult somatic cells.^{38, 39, 40}

“Immune response” broadly refers to the antigen-specific responses of lymphocytes to foreign substances. The terms “immunogen” and “immunogenic” refer to molecules with the capacity to elicit an immune response. All immunogens are antigens, however, not all antigens are immunogenic. An immune response disclosed herein can be humoral (via antibody activity) or cell-mediated (via T cell activation). The response may occur in vivo or in vitro. The skilled artisan will understand that a variety of macromolecules, including proteins, nucleic acids, fatty acids, lipids, lipopolysaccharides and polysaccharides have the potential to be immunogenic. The skilled artisan will further understand that nucleic acids encoding a molecule capable of eliciting an immune response necessarily encode an immunogen. The artisan will further understand that immunogens are not limited to full-length molecules, but may include partial molecules.

As used herein, the term “inducing an immune response in a subject” or “modulating an immune response” are terms well understood in the art and intends that an increase or decrease of at least about 2-fold, at least about 5-fold, at least about 10-fold, at least about 100-fold, at least about 500-fold, or at least about 1000-fold or more in an immune response (i.e. T cell or antibody response) to an antigen (or epitope) and can be detected or measured by various methods known in the art. For example, the frequency or activity of antigen-specific T cells can be measured by multiple methods, including, but not limited to, flow cytometry, RNA-sequencing or in vitro assays.

As used herein, the term “modulating activity” refers to increasing or decreasing the activity of specific T cell populations associated with an immune response. Modulating of activity may be accomplished by the administration of agents, including antibodies, that target and bind to specific T cell receptors in order to activate the T cell population expressing that molecule. Modulation may occur when the T cells are engaged by costimulatory ligands, agonistic antibodies or cytokines. In some aspects, modulating activity may include the administration of an agent that targets a molecule on a T cell. In some aspects, the molecule is JAML and the agent is an antibody that targets JAML, thus activating the JAML expressing T cell.

An “immunotherapy agent” means a type of cancer treatment which uses a patient's own immune system to fight cancer, including but not limited to a physical intervene, a chemical substance, a biological molecule or particle, a cell, a tissue or organ, or any combinations thereof, enhancing or activating or initiating a patient's immune response against cancer. Non-limiting examples of immunotherapy agents include antibodies, immune regulators, checkpoint inhibitors, an antisense oligonucleotide (ASO), a RNA interference (RNAi), a Clustered Regularly Interspaced Short Palindromic Repeat (CRISPR) system, a viral vector, an anti-cancer cell therapy (e.g., transplanting an anti-cancer immune cell optionally amplified and/or activated in vivo, or administering an immune cell expressing a chimeric antigen receptor (CAR)), a CAR therapy, and cancer vaccines.

As used herein, immune checkpoint refers to a regulator and/or modulator of the immune system (such as an immune response, an anti-tumor immune response, a nascent anti-tumor immune response, an anti-tumor immune cell response, an anti-tumor T cell response, and/or an antigen recognition of T cell receptor in the process of immune response). Their interaction activates either inhibitory or activating immune signaling pathways. Thus a checkpoint may contain one of the two signals: an stimulatory immune checkpoint that stimulates an immune response, and an inhibitory immune checkpoint inhibiting an immune response. In some embodiments, the immune checkpoint is crucial for self-tolerance, which prevents the immune system from attacking cells indiscriminately. However, some cancers can protect themselves from attack by stimulating immune checkpoint targets. In some embodiments, the immune checkpoints are present on T cells, antigen-presenting cells (APCs) and/or tumor cells.

A “composition” is intended to mean a combination of active polypeptide, polynucleotide or antibody and another compound or composition, inert (e.g., a detectable label) or active (e.g., a gene delivery vehicle).

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

An agent of the present disclosure can be administered for therapy by any suitable route of administration. It will also be appreciated that the optimal route will vary with the condition and age of the recipient, and the disease being treated.

The term “effective amount” refers to a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. With respect to immunogenic compositions, in some embodiments the effective amount will depend on the intended use, the degree of immunogenicity of a particular antigenic compound, and the health/responsiveness of the subject's immune system, in addition to the factors described above. The skilled artisan will be able to determine appropriate amounts depending on these and other factors.

In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

“Simultaneous use” as used herein refers to the administration of the two compounds of the composition according to the invention in a single and identical pharmaceutical form or at the same time in two distinct pharmaceutical forms.

“Separate use” as used herein refers to the administration, at the same time, of the two compounds of the composition according to the invention in distinct pharmaceutical forms.

“Sequential use” as used herein refers to the successive administration of the two compounds of the composition according to the invention, each in a distinct pharmaceutical form.

In one embodiment, the therapy is combined with a chemotherapeutic agent. A “chemotherapeutic agent,” as used herein, refers to a substance which, when administered to a subject, treats or prevents the development of cancer in the subject's body. Chemotherapeutic agents include, but are not limited to, alkylating agents, anti-metabolites, anti-tumor antibiotics, mitotic inhibitors, chromatin function inhibitors, anti-angiogenesis agents, anti-estrogens, anti-androgens or immunomodulators.

“Alkylating agent” refers to any substance which can cross-link or alkylate any molecule, preferably nucleic acid (e.g., DNA), within a cell. Examples of alkylating agents include nitrogen mustard such as mechlorethamine, chlorambucol, melphalen, chlorydrate, pipobromen, prednimustin, disodic-phosphate or estramustine; oxazophorins such as cyclophosphamide, altretamine, trofosfamide, sulfofosfamide or ifosfamide; aziridines or imine-ethylenes such as thiotepa, triethylenamine or altetramine; nitrosourea such as carmustine, streptozocin, fotemustin or lomustine; alkyle-sulfonates such as busulfan, treosulfan or improsulfan; triazenes such as dacarbazine; or platinum complexes such as cis-platinum, oxaliplatin and carboplatin.

“Anti-metabolites” refer to substances that block cell growth and/or metabolism by interfering with certain activities, usually DNA synthesis. Examples of anti-metabolites include methotrexate, 5-fluoruracil, floxuridine, 5-fluorodeoxyuridine, capecitabine, cytarabine, fludarabine, cytosine arabinoside, 6-mercaptopurine (6-MP), 6-thioguanine (6-TG), chlorodesoxyadenosine, 5-azacytidine, gemcitabine, cladribine, deoxycoformycin and pentostatin.

“Anti-tumor antibiotics” refer to compounds which may prevent or inhibit DNA, RNA and/or protein synthesis. Examples of anti-tumor antibiotics include doxorubicin, daunorubicin, idarubicin, valrubicin, mitoxantrone, dactinomycin, mithramycin, plicamycin, mitomycin C, bleomycin, and procarbazine.

“Mitotic inhibitors” prevent normal progression of the cell cycle and mitosis. In general, microtubule inhibitors or taxoides such as paclitaxel and docetaxel are capable of inhibiting mitosis. Vinca alkaloid such as vinblastine, vincristine, vindesine and vinorelbine are also capable of inhibiting mitosis.

“Chromatin function inhibitors” or “topoisomerase inhibitors” refer to substances which inhibit the normal function of chromatin modeling proteins such as topoisomerase I or topoisomerase II. Examples of chromatin function inhibitors include, for topoisomerase I, camptothecine and its derivatives such as topotecan or irinotecan, and, for topoisomerase II, etoposide, etoposide phosphate and teniposide.

“Anti-angiogenesis agent” refers to any drug, compound, substance or agent which inhibits growth of blood vessels. Exemplary anti-angiogenesis agents include, but are by no means limited to, razoxin, marimastat, batimastat, prinomastat, tanomastat, ilomastat, CGS-27023A, halofuginon, COL-3, neovastat, BMS-275291, thalidomide, CDC 501, DMXAA, L-651582, squalamine, endostatin, SU5416, SU6668, interferon-alpha, EMD121974, interleukin-12, IM862, angiostatin and vitaxin.

“Anti-estrogen” or “anti-estrogenic agent” refer to any substance which reduces, antagonizes or inhibits the action of estrogen. Examples of anti-estrogen agents are tamoxifen, toremifene, raloxifene, droloxifene, iodoxyfene, anastrozole, letrozole, and exemestane.

“Anti-androgens” or “anti-androgen agents” refer to any substance which reduces, antagonizes or inhibits the action of an androgen. Examples of anti-androgens are flutamide, nilutamide, bicalutamide, sprironolactone, cyproterone acetate, finasteride and cimitidine.

“Immunomodulators” are substances which stimulate the immune system.

Examples of immunomodulators include interferon, interleukin such as aldesleukine, OCT-43, denileukin diflitox and interleukin-2, tumoral necrosis factors such as tasonermine or others immunomodulators such as lentinan, sizofiran, roquinimex, pidotimod, pegademase, thymopentine, poly I:C or levamisole in conjunction with 5-fluorouracil.

Chemical agents or cytotoxic agents include all kinase inhibitors such as, for example, gefitinib or erlotinib.

More generally, examples of suitable chemotherapeutic agents include but are not limited to 1-dehydrotestosterone, 5-fluorouracil decarbazine, 6-mercaptopurine, 6-thioguanine, actinomycin D, adriamycin, aldesleukin, alkylating agents, allopurinol sodium, altretamine, amifostine, anastrozole, anthramycin (AMC)), anti-mitotic agents, cis-dichlorodiamine platinum (II) (DDP) cisplatin), diamino dichloro platinum, anthracyclines, antibiotics, antimetabolites, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunirubicin HCL, daunorucbicin citrate, denileukin diftitox, Dexrazoxane, Dibromomannitol, dihydroxy anthracin dione, Docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, oxaliplatin, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, tegafur, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

5-Fluorouracil (5-FU) belongs to the family of therapy drugs called pyrimidine based anti-metabolites. It is a pyrimidine analog, which is transformed into different cytotoxic metabolites that are then incorporated into DNA and RNA thereby inducing cell cycle arrest and apoptosis. Chemical equivalents are pyrimidine analogs which result in disruption of DNA replication. Chemical equivalents inhibit cell cycle progression at S phase resulting in the disruption of cell cycle and consequently apoptosis. Equivalents to 5-FU include prodrugs, analogs and derivative thereof such as 5′-deoxy-5-fluorouridine (doxifluoroidine), 1-tetrahydrofuranyl-5-fluorouracil (ftorafur), capecitabine (Xeloda®), S-1 (MBMS-247616, consisting of tegafur and two modulators, a 5-chloro-2,4-dihydroxypyridine and potassium oxonate), ralititrexed (tomudex), nolatrexed (Thymitaq, AG337), LY231514 and ZD9331, as described for example in Papamichael (1999) The Oncologist 4:478-487.

“5-FU based adjuvant therapy” refers to 5-FU alone or alternatively the combination of 5-FU with one or more other treatments, that include, but are not limited to radiation, methyl-CCNU, leucovorin, oxaliplatin (such as cisplatin), irinotecan, mitomycin, cytarabine, doxorubicin, cyclophosphamide, and levamisole, as well as an immunotherapy. Specific treatment adjuvant regimens are known in the art such as weekly Fluorouracil/Leucovorin, weekly Fluorouracil/Leucovorin+Bevacizumab, FOLFOX, FOLFOX-4, FOLFOX6, modified FOLFOX6 (mFOLFOX6), FOLFOX6 with bevacizumab, mFOLFOX6+Cetuximab, mFOLFOX6+Panitumumab, modified FOLFOX7 (mFOLFOX7), FOLFIRI, FOLFIRI with Bevacizumab, FOLFIRI+Ziv-aflibercept, FOLFIRI with Cetuximab, FOLFIRI+Panitumumab, FOLFIRI+Ramucirumab, FOLFOXIRI, FOLFIRI with FOLFOX6, FOLFOXIRI+Bevacizumab, FOLFOXIRI+Cetuximab, FOLFOXIRI+Panitumumab, Roswell Park Fluorouracil/Leucovorin, Roswell Park Fluorouracil/Leucovorin+Bevacizumab, Simplified Biweekly Infusional Fluorouracil/Leucovorin, Simplified Biweekly Infusional Fluorouracil/Leucovorin+Bevacizumab, and MOF (semustine (methyl-CCNU), vincrisine (Oncovin®) and 5-FU). For a review of these therapies see Beaven and Goldberg (2006) Oncology 20(5):461-470 as well as www.cancertherapyadvisor.com/home/cancer-topics/gastrointestinal-cancers/gastrointestinal-cancers-treatment-regimens/colon-cancer-treatment-regimens/. Other chemotherapeutics can be added, e.g., oxaliplatin or irinotecan.

Capecitabine is a prodrug of (5-FU) that is converted to its active form by the tumor-specific enzyme PynPase following a pathway of three enzymatic steps and two intermediary metabolites, 5′-deoxy-5-fluorocytidine (5′-DFCR) and 5′-deoxy-5-fluorouridine (5′-DFUR). Capecitabine is marketed by Roche under the trade name Xeloda®.

Leucovorin (Folinic acid) is an adjuvant used in cancer therapy. It is used in synergistic combination with 5-FU to improve efficacy of the chemotherapeutic agent. Without being bound by theory, addition of Leucovorin is believed to enhance efficacy of 5-FU by inhibiting thymidylate synthase. It has been used as an antidote to protect normal cells from high doses of the anticancer drug methotrexate and to increase the antitumor effects of fluorouracil (5-FU) and tegafur-uracil. It is also known as citrovorum factor and Wellcovorin. This compound has the chemical designation of L-Glutamic acid N-[4-[[(2-amino-5-formyl-1,4,5,6,7,8-hexahydro-4-oxo-6-pteridinyl)methyl]amino]benzoyl], calcium salt (1:1).

“Oxaliplatin” (Eloxatin) is a platinum-based chemotherapy drug in the same family as cisplatin and carboplatin. It is typically administered in combination with fluorouracil and leucovorin in a combination known as FOLFOX for the treatment of colorectal cancer. Compared to cisplatin, the two amine groups are replaced by cyclohexyldiamine for improved antitumor activity. The chlorine ligands are replaced by the oxalato bidentate derived from oxalic acid in order to improve water solubility. Equivalents to Oxaliplatin are known in the art and include, but are not limited to cisplatin, carboplatin, aroplatin, lobaplatin, nedaplatin, and JM-216 (see McKeage et al. (1997) J. Clin. Oncol. 201:1232-1237 and in general, Chemotherapy for Gynecological Neoplasm, Curr. Therapy and Novel Approaches, in the Series Basic and Clinical Oncology, Angioli et al. Eds., 2004).

“FOLFOX” is an abbreviation for a type of combination therapy that is used to treat cancer. This therapy includes leucovorin (“FOL”), 5-FU (“F”), and oxaliplatin (“OX”) and encompasses various regimens, such as FOLFOX-4, FOLFOX-6, modified FOLOX-6, and FOLFOX-7, which vary in doses and ways in which each of the three drugs are administered. “FOLFIRI” is an abbreviation for a type of combination therapy that is used treat cancer and comprises, or alternatively consists essentially of, or yet further consists of 5-FU, leucovorin, and irinotecan. Information regarding these treatments are available on the National Cancer Institute's web site, cancer.gov, last accessed on Oct. 28, 2021.

Irinotecan (CPT-11) is sold under the trade name of Camptosar. It is a semi-synthetic analogue of the alkaloid camptothecin, which is activated by hydrolysis to SN-38 and targets topoisomerase I. Chemical equivalents are those that inhibit the interaction of topoisomerase I and DNA to form a catalytically active topoisomerase I-DNA complex. Chemical equivalents inhibit cell cycle progression at G2-M phase resulting in the disruption of cell proliferation.

S-1 consists of three agents (at a molar ratio of 1:0.4:1): tegafur, 5-chloro-2-4-dihydroxypyridine, and potassium oxonate.

The term “adjuvant” therapy refers to administration of a therapy or chemotherapeutic regimen to a patient in addition to the primary or initial treatment, such as after removal of a tumor by surgery. Adjuvant therapy is typically given to minimize or prevent a possible cancer reoccurrence. Alternatively, “neoadjuvant” therapy refers to administration of therapy or chemotherapeutic regimen before surgery, typically in an attempt to shrink the tumor prior to a surgical procedure to minimize the extent of tissue removed during the procedure. Additionally or alternatively, such adjuvant therapy potentials (i.e., sensitizes the subject to the original therapy) the subject may help reach one or more of clinical end points of the cancer treatment.

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected) or a group of tissues making up an organ or part or region of the body of an organism. The tissue may comprise a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, the term “treatment” excludes prevention.

### Modes for Carrying Out the Disclosure

### Therapeutic Methods

Provided herein are methods for one or more of a) modulating an immune response to a tumor cell or cancer cell in a patient, b) treating cancer in a cancer patient; or c) eliciting an anti-tumor or anti-cancer response in a patient. The method comprises, consists of or consists essentially of modulating the expression or activity of Junction Adhesion Molecule Like (JAML). In some aspects, the modulation of JAML comprises, consists of, or consists essentially of activating the T cell by agonizing the expression or activity of JAML. In one aspect, JAML is expressed on an immune cell such as for example, a T cell.

In one aspect of this disclosure, the expression or activity of JAML is modulated by administering an effective amount of an agent that targets JAML in the T cell. In some aspects, the T cell is selected from the group of: In some aspects, the T cell is selected from the group of: an activated T cell, a tissue resident memory (TRM) cell, CD8+ T cell, an αβ CD8⁺ T cell, or a stem T cell. In some aspects, the activated T cell is specific for a tumor-associated antigen expressed by the tumor cells that is optionally overexpressed or specifically expressed by the tumor cell.

In some aspects of this disclosure, the agent that targets JAML in the T cell binds to JAML and a second molecule expressed by the T cell. In some aspects, the second molecule is selected from the group of CXCR5, CXCR6, CD8, CD103, CD49A, CD69, CD3, or PD-1. In some aspects, the second molecule comprises, consists of, or consists essentially of CXCR5.

In yet another aspect, the agent that binds to JAML comprises, consists of, or consists of an agonistic antibody targeting JAML and thus activates or augments JAML activity or expression in the T cell. In some aspects, the agent comprises, consists of, or consists essentially of a bispecific antibody that binds to JAML and a second molecule expressed by the T cell. The bispecific antibodies of the present disclosure provide further specificity for identifying JAML expressing T cells in order to avoid undesirable off-target antibody activity. Thus, in some aspects, the bispecific antibodies only activate T cells expressing both JAML and the second molecule expressed by the T cell. In some aspects, the T cell is selected from the group of: an activated T cell, a tissue resident memory (TRM) cell, CD8+ T cell, an αβ CD8⁺ T cell, or a stem T cell.

In yet another aspect of the disclosure, the agent binds to JAML and binds to a tumor or cancer antigen expressed by the tumor or cancer cell that is optionally overexpressed or specifically expressed by the tumor or cancer cell. In some aspects, the tumor antigen comprises, consists of, or consists essentially of a tumor associated antigen specifically expressed by the tumor cell. In some aspects, the tumor antigen is overexpressed by the tumor cell as compared to the expression in a normal counterpart cell. In some aspects, the tumor antigen is selected from the group of: a cancer testis antigen or a cancer embryonic antigen (CEA). In some aspects, the tumor antigen is selected from the group of: MAGE-D4B, PSMA, HER₂, HER₃, EGFR, AFP, CEA, CA-125, MUC-1, ETA, MUC-1, BAGE, GAGE-1, MAGE-A1, NY-ESO-1, Gp100, Melan-A/MART-1, Prostate-specific antigen, Mammoglobin-A, Alpha-fetoprotein, HER-2/neu, P53, K-ras, or TRP-2/INT2. In some aspects, the tumor antigen comprises, consists of, or consists essentially of a tumor antigen that has yet to be identified. In some aspects, the agent comprises, consists of, or consists essentially of a bispecific antibody that binds to JAML and the tumor antigen.

In some aspects of this disclosure, the cancer or tumor is a cancer of at least one of the following organs: an epithelial, a head, neck, lung, prostate, colon, breast, testis, bone, lymphatic system, blood, endometrium, uterus, ovary, pancreas, esophagus, liver, skin, kidney, adrenal gland, brain. The cancer can be from the group of; a lymphoma, leukemia, breast cancer, endometrial cancer, uterine, ovarian cancer, testicular cancer, lung cancer, prostate cancer, colon cancer, rectal cancer pancreatic cancer, esophageal cancer, liver cancer, melanoma, or other skin cancers, kidney cancer, adrenal gland cancer, a non-small cell lung cancer (NSCLC) and/or head and neck squamous cell cancer (HNSCC) and/or brain cancer or tumor. It can be of any stage (primary or metastatic) or a recurring tumor or cancer or neoplasia.

In some aspects, the patient is a mammal such as for example, a human patient.

In some aspects of this disclosure, the methods further comprise, consist of, or consist essentially of resecting the tumor or cancer prior to modulating the expression or activity of JAML in the T cell in the patient. In some aspects, the T cell is selected from the group of: an activated T cell, a tissue resident memory (TRM) cell, CD8+ T cell, an up CD8⁺ T cell, or a stem T cell. In some aspects, the modulating expression or activity of JAML in a T cell is administered as a first-line, a second-line, a third-line, a fourth line or fifth line therapy.

In some aspects of this disclosure, the methods further comprise, consist of, or consist essentially of administering an effective amount of an anti-cancer agent to the patient. Non-limiting examples of such are provided herein.

In some aspects of this disclosure, the patient being treated experiences one or more of a reduction in tumor burden, longer overall survival or prolonged time to tumor progression.

In yet another aspect of this disclosure, provided herein is a method for screening for a JAML anticancer therapy comprising, consisting of, or consisting essentially of contacting a first sample of T cells with an amount of the test agent that binds to JAML, and assaying for increased expression of JAML in the T cell. In some aspects, increased expression of JAML in the T cell is an indication that the agent is a JAML anticancer therapy. The T cells can be from patient biopsies or can be commercially obtained or cultured cells. In some aspects, the T cell is selected from the group of: an activated T cell, a tissue resident memory (TRM) cell, CD8+ T cell, an αβ CD8⁺ T cell, or a stem T cell. In some aspects, the T cell is a stem T cell.

In other aspects, the test agent can be selected for sample can further comprise molecule that targets a cancer or tumor cell and the agent to be tested is specific for JAML and cancer or tumor cell.

In yet another aspect of this disclosure, provided herein is a method for screening for a JAML anticancer therapy comprising, consisting of, or consisting essentially of contacting a first sample of T cells with an amount of the test agent that binds to JAML and a cancer or tumor antigen, and assaying for increased expression of JAML in the T cell. In some aspects, increased expression of JAML in the T cell is an indication that the agent is a JAML anticancer therapy. The sample of T cells can further comprise the cancer or tumor cell being targeted by the second agent and they can be from patient biopsies or can be commercially obtained or cultured cells. In some aspects, the T cell is selected from the group of: an activated T cell, a tissue resident memory (TRM) cell, CD8+ T cell, an αβ CD8⁺ T cell, or a stem T cell. The T cell can be from patient biopsies or can be commercially obtained or cultured cells.

In yet another aspect of this disclosure, provided herein is a method of modulating JAML in a T cell in vitro or in a subject comprising, consisting of, or consisting essentially of contacting the T cell in vitro or by administering a bispecific antibody that targets and binds to JAML and a molecule expressed by a T cell. In some aspects, the molecule expressed by the T cell is selected from CXCR5, CXCR6, CD8, CD103, CD49A, CD69, CD3, or PD-1. In some aspects, the T cell is selected from the group of: an activated T cell, a tissue resident memory (TRM) cell, CD8+ T cell, an αβ CD8⁺ T cell, or a stem T cell. In some aspects, the T cell is a stem T cell.

In yet another aspect of this disclosure, provided herein is a method of diagnosing cancer in a subject by contacting a sample isolated from the subject with an agent that detects the presence of JAML or CXADR in the sample isolated from the subject. In some aspects, the presence of JAML or CXADR at higher or lower than baseline expression levels is diagnostic of cancer.

In yet another aspect of this disclosure, provided herein is a method of diagnosing cancer in a subject comprising, consisting of, or consisting essentially of contacting T cells isolated from the subject or tissue or cells suspected of containing cancer isolated from the subject, with an antibody or agent that recognizes and binds to JAML. If the agent binds to the cells, tissue or sample, the subject likely has cancer.

In yet another aspect of this disclosure, provided herein is a method of determining prognosis of a subject having cancer comprising, consisting of, or consisting essentially of measuring the density of CXADR expressing cells in a sample isolated from the subject, wherein a low density of cells indicates a more positive prognosis or wherein a high density of cells indicates a more negative prognosis, optionally wherein the more negative prognosis comprises a decreased probability of survival, and wherein the more positive prognosis comprises an increased probability of survival.

In yet another aspect of this disclosure, provided herein is a method of determining prognosis of a subject having cancer the method comprising, consisting of, or consisting essentially of contacting T cells isolated from the subject with an antibody or agent that recognizes and binds to JAML to determine the frequency of T cells expressing JAML in tumor cells, wherein a high frequency of JAML in T cells indicates a more positive prognosis or wherein a low frequency of JAML in T cells indicates a more negative prognosis, optionally wherein the more negative prognosis comprises a decreased probability of survival, and wherein the more positive prognosis comprises an increased probability of survival.

In yet another aspect of this disclosure, provided herein is a method of determining the responsiveness of a cancer subject to cancer therapy, the method comprising, consisting of, or consisting essentially of contacting T cells isolated from the subject with an antibody or agent that recognizes and binds to JAML to determine the frequency of JAML expressing T cells in the subject, wherein a high frequency of JAML T cells indicates an increased likelihood of responsiveness to a cancer therapy. In some aspects, the sample comprises, consists of, or consists essentially of a tumor sample. In some aspects, the cancer therapy comprises, consists of, or consists essentially of an agent that modulates the expression and/or activity of JAML in the subject.

In yet another aspect of this disclosure, provided herein is a method of identifying a cancer subject that is likely to respond to a cancer therapy, comprising, consisting of, or consisting essentially of contacting a sample isolated from the subject with an agent that detects the presence of CXADR in the sample, wherein the presence of CXADR at lower than baseline expression levels indicates that the subject is likely to respond to the cancer therapy.

In some aspects of these methods, the agent that binds to JAML and/or the T cell or cancer or tumor cell can be detectably labeled or tagged. In some aspects, the detectable label or tag comprises, consists of, or consist essentially of a radioisotope, a metal, horseradish peroxidase, alkaline phosphatase, avidin or biotin.

In some aspects, baseline expression comprises, consists of, or consists essentially of normalized mean expression. In some aspects, higher than baseline expression of CXADR or JAML comprises, consists of, or consists essentially of at least about a 2-fold increase in expression relative to baseline expression and/or lower than baseline expression of CXADR or JAML is at least about a 2-fold decrease in expression relative to baseline expression.

In some aspects, the methods provided herein further comprise, consist of, or consist essentially of administering a cancer therapy to the subject. In some aspects, the cancer therapy comprises, consists of, or consists essentially of an agent that binds to JAML. In some aspects, the agent comprises, consists of, or consists essentially of an agonistic antibody targeting JAML.

In some aspects, the sample for use in the methods comprises, consists of, or consists essentially of cells, tissue, an organ biopsy, an epithelial tissue, a lung, respiratory or airway tissue or organ, a circulatory tissue or organ, a skin tissue, bone tissue, muscle tissue, head, neck, brain, skin, bone and/or blood sample.

In some aspects, the agent comprises, consists of, or consists essentially of a polypeptide that binds to an expression product encoded by JAML, or a polynucleotide that hybridizes to a nucleic acid sequence encoding all or a portion of JAML. In some aspects, the polypeptide comprises, consists of, or consists essentially of an antibody, an antigen binding fragment thereof, or a receptor that binds to the JAML. In some aspects, the antibody comprises, consists of, or consists essentially of an IgG, IgA, IgM, IgE or IgD, or a subclass thereof. In some aspects, the IgG comprises, consists of, or consists essentially of an IgG1, IgG2, IgG3 or IgG4. In some aspects, the antigen binding fragment comprises, consists of, or consists essentially of a Fab, Fab′, F(ab′)₂, Fv, Fd, single-chain Fvs (scFv), disulfide-linked Fvs (sdFv) or VL or VH. In some aspects, the agent is contacted with the sample in conditions under which it can bind to the JAML.

Binding of agents to the cells can be detected by methods known in the art, and described herein. Non-limiting examples of the therapeutic, diagnostic, and prognostic methods described herein include for example, detection by immunohistochemistry (IHC), in-situ hybridization (ISH), ELISA, immunoprecipitation, immunofluorescence, chemiluminescence, radioactivity, X-ray, nucleic acid hybridization, protein-protein interaction, immunoprecipitation, flow cytometry, Western blotting, polymerase chain reaction, DNA transcription, Northern blotting and/or Southern blotting. To facilitate detection, the test agents can be detectably labeled or tagged.

Also provided herein are methods for one or more, two or more, three or more, four or more, five or more, or six or more of: inhibiting activation of checkpoint protein expressing follicular regulatory T (T_{FR}) cells; augmenting tumor infiltrating lymphocytes (TILs); augmenting checkpoint inhibitor therapy; eliciting an anti-cancer response against a cancer expressing a checkpoint protein; inhibiting the growth of cancer cells expressing a checkpoint protein; or treating a cancer expressing a checkpoint protein. The methods comprise or consist essentially of, or yet further consist of administering to a subject in need thereof a therapy comprising an effective amount of an agonistic anti-junctional adhesion molecule-like protein (JAML) antibody and a checkpoint inhibitor therapy. Applicants have found that combination of the anti-JAML antibody inhibits activation of checkpoint expressing T_{FR} cells in a subject receiving the therapy as compared to the T_{FR} cells in a subject not receiving the therapy. The administration of the anti-JAML therapy augments the effectiveness of checkpoint inhibitor therapy. The administration of the anti-JAML therapy and checkpoint inhibitor therapy elicits an anti-cancer response against a cancer expressing a checkpoint protein; inhibits the growth of cancer cells expressing a checkpoint protein, treats a cancer expressing a checkpoint protein, and/or augments tumor infiltrating lymphocytes (TILs) in the subject.

The subject to be treated has cancer or is at high risk of cancer, recurrence or disease progression. The subject can be an animal such as a mammal or a human patient.

Administration can be effected in any appropriate manner as determined by the treating physician or veterinarian, and the amount will vary with the subject being treated, the cancer, the age and general health and well-being of the subject.

Anti-JAML antibodies for administration include monoclonal, polyclonal, antigen binding fragments, derivative and modifications thereof as known in the art and described herein.

In one aspect, the checkpoint inhibitor comprises, consists of, or consists essentially of GS4224, AMP-224, CA-327, CA-170, BMS-1001, BMS-1166, peptide-57, M7824, MGD013, CX-072, UNP-12, NP-12, or a combination of two or more thereof.

In one aspect of the method, the checkpoint inhibitor comprises one or more selected from an anti-PD-1 agent, an anti-PD-L1 agent, an anti-CTLA-4 agent, an anti-LAG-3 agent, an anti-TIM-3 agent, an anti-TIGIT agent, an anti-VISTA agent, an anti-B7-H3 agent, an anti-BTLA agent, an anti-ICOS agent, an anti-GITR agent, an anti-4-1BB agent, an anti-OX40 agent, an anti-CD27 agent, an anti-CD28 agent, an anti-CD40 agent, and an anti-Siglec-15 agent. In another aspect, the checkpoint inhibitor comprises an anti-PD1 agent or an anti-PD-L1 agent. In a further aspect, the anti-PD1 agent comprises an anti-PD1 antibody or an antigen binding fragment thereof. Non-limiting examples of an anti-PD1 antibody comprises nivolumab, pembrolizumab, cemiplimab, spartalizumab, camrelizumab, sintilimab, tislelizumab, toripalimab, AMF 514, or a combination of two or more thereof. In one embodiment, the anti-PD-L1 agent comprises an anti-PD-L1 antibody or an antigen binding fragment thereof. In another aspect, the anti-PD-L1 antibody comprises avelumab, durvalumab, atezolizumab, envafolimab, or a combination of two or more thereof. In another embodiment, the checkpoint inhibitor comprises an anti-CTLA-4 agent. In one aspect, the anti-CTLA-4 agent comprises an anti-CTLA-4 antibody or an antigen binding fragment thereof. In a further embodiment, the anti-CTLA-4 antibody comprises ipilimumab, tremelimumab, zalifrelimab, or AGEN1181, or a combination thereof. In one embodiment, the checkpoint inhibitor comprises an anti-PD1 agent or an anti-PD-L1 agent and an anti-CTLA-4 agent. In another embodiment, the anti-PD1 agent comprises an anti-PD1 antibody or an antigen binding fragment thereof. Non-limiting examples of the anti-PD1 antibody comprises nivolumab, pembrolizumab, cemiplimab, spartalizumab, camrelizumab, sintilimab, tislelizumab, toripalimab, AMF 514, or a combination of two or more thereof. Alternatively, the anti-PD-L1 agent comprises an anti-PD-L1 antibody or an antigen binding fragment thereof, and non-limiting examples thereof include avelumab, durvalumab, atezolizumab, envafolimab, or a combination of two or more thereof. In another aspect, the anti-CTLA-4 agent comprises an anti-CTLA-4 antibody or an antigen binding fragment thereof, and non-limiting examples of such include ipilimumab, tremelimumab, zalifrelimab, or AGEN1181, or a combination thereof.

The agents can be combined and administered concurrently or sequentially in another appropriate order.

The methods are useful for cancers of the following organs or systems: circulatory system; respiratory tract; gastrointestinal system genitourinary tract; live; bone; nervous system; reproductive system; hematologic system; oral cavity; skin and other tissues comprising connective and soft tissue, retroperitoneum and peritoneum, eye, intraocular melanoma, and adnexa, breast, head or/and neck, anal region, thyroid, parathyroid, adrenal gland and other endocrine glands and related structures, and lymph nodes, optionally wherein the cancer is a solid tumor or alternatively wherein the cancer is a liquid cancer, and further optionally wherein the cancer is a primary cancer or a metastasis. In one aspect, the cancer comprises a carcinoma, a sarcoma, a myeloma, a leukemia, or a lymphoma. In a further aspect, the carcinoma is selected from a colon cancer, a rectal cancer, a colorectal cancer, a breast cancer, a colon carcinoma, a lunch cancer, a small cell lung cancer, a non-small cell lung cancer, a head and neck squamous carcinoma, or a melanoma. In a yet further aspect, the sarcoma is selected from an angiosarcoma, a chondrosarcoma, a Ewing sarcoma, a leiomyosarcoma, a malignant peripheral nerve sheath tumor, an osteosarcoma, a rhabdomyosarcoma, a synovial sarcoma, a dedifferentiated liposarcoma, or a gastrointestinal stromal tumor.

The therapy can be administered as a first line therapy, a second line therapy, a third line therapy, a fourth line therapy, or a fifth line therapy. In a further aspect, the method further comprises administering to the subject an effective amount of a cytoreductive therapy, for example, one or more of chemotherapy, immunotherapy, or radiation therapy.

In another aspect, the method further comprising determining if the cancer expresses a checkpoint protein, and optionally identifying the checkpoint protein expressed by the cancer cell or tumor. This diagnostic method can be performed or after administration of the therapy. In a further aspect, the checkpoint inhibitor therapy is selected to target the checkpoint protein, e.g., the cell expresses PD-1 and an anti-PD-1 or PD-L1 therapy is administered.

Successful therapy can be determined by any appropriate criteria, e.g., if the subject experiences one or more endpoints selected from tumor response, reduction in tumor size, reduction in tumor burden, increase in overall survival, increase in progression free survival, inhibiting metastasis, improvement of quality of life, minimization of toxicity, and avoidance of side-effects.

### Therapeutic Compositions and Combinations

This disclosure also provide a composition or combination of active agents comprising, or consisting essentially of, or yet further consisting of an anti-junctional adhesion molecule-like protein (JAML) antibody or JAML binding fragment thereof and a checkpoint inhibitor therapy.

Also provided is a composition or combination of active agents comprising, or consisting essentially of, or yet further consisting of an agonistic anti-junctional adhesion molecule-like protein (JAML) antibody or JAML binding fragment thereof and a checkpoint inhibitor therapy.

The active agents can further comprise an additional therapeutic agent (examples of such are described herein) a carrier such as a pharmaceutically acceptable carrier and can be formulated in combination or separately, for concurrent or sequential administration.

In one aspect, the compositions are formulated with one or more pharmaceutically acceptable excipients, diluents, carriers and/or adjuvants. In addition, embodiments of the compositions of the present disclosure include one or more of an isolated polypeptide disclosed herein, an isolated polynucleotide disclosed herein, a vector disclosed herein, a small molecule, an isolated host cell disclosed herein, or an antibody of the disclosure, formulated with one or more pharmaceutically acceptable substances.

For oral preparations, any one or more of an isolated or recombinant polypeptide as described herein, an isolated or recombinant polynucleotide as described herein, a vector as described herein, an isolated host cell as described herein, a small molecule or an antibody as described herein can be used alone or in pharmaceutical formulations disclosed herein comprising, or consisting essentially of, the compound in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical formulations and unit dose forms suitable for oral administration are particularly useful in the treatment of chronic conditions, infections, and therapies in which the patient self-administers the drug. In one aspect, the formulation is specific for pediatric administration.

Aerosol formulations provided by the disclosure can be administered via inhalation and can be propellant or non-propellant based. For example, embodiments of the pharmaceutical formulations disclosed herein comprise a compound disclosed herein formulated into pressurized acceptable propellants such as dichlorodifluoromethane, propane, nitrogen and the like. For administration by inhalation, the compounds can be delivered in the form of an aerosol spray from a pressurized container or dispenser which contains a suitable propellant, e.g., a gas such as carbon dioxide, or a nebulizer. A non-limiting example of a non-propellant is a pump spray that is ejected from a closed container by means of mechanical force (i.e., pushing down a piston with one's finger or by compression of the container, such as by a compressive force applied to the container wall or an elastic force exerted by the wall itself, e.g., by an elastic bladder).

Suppositories disclosed herein can be prepared by mixing an active agent disclosed herein with any of a variety of bases such as emulsifying bases or water-soluble bases. Embodiments of this pharmaceutical formulation of a compound disclosed herein can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more the active agents disclosed herein. Similarly, unit dosage forms for injection or intravenous administration may comprise an active agent disclosed herein in a composition as a solution in sterile water, normal saline or another pharmaceutically acceptable carrier.

The composition or combination can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of a compound disclosed herein can be accomplished using any of a variety of refillable, pump systems. Pumps provide consistent, controlled release over time. In some embodiments, a compound disclosed herein is in a liquid formulation in a drug-impermeable reservoir, and is delivered in a continuous fashion to the individual.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852; and the like. In general, a subject treatment method can be accomplished using any of a variety of refillable, non-exchangeable pump systems. Pumps and other convective systems may be utilized due to their generally more consistent, controlled release over time. Osmotic pumps are used in some embodiments due to their combined advantages of more consistent controlled release and relatively small size (see, e.g., PCT International Application Publication No. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like. A further exemplary device that can be adapted for the present disclosure is the Synchromed infusion pump (Medtronic).

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

Suitable excipient vehicles for a compound disclosed herein are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Compositions of the present disclosure include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylatanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

Anti-JAML antibodies for the combination or composition include monoclonal, polyclonal, antigen binding fragments, derivative and modifications thereof as known in the art and described herein. Exemplary anti-JAML antibodies are commercially available and described herein, e.g., https://www.biolegend.com/fr-fr/products/purified-anti-mouse-jaml-antibody-5050, https://www.abeam.com/products?sortOptions=Relevance&selected.classification=Primary+anti bodies&keywords=JAML&gclid=CjwKCAjw5P2aBhAlEiwAAdY7dGA1Rd_Rca2c1dYh2X7A MZKZxmHMkrl3AWNZa2wzSKNLoy9vOjCo9xoC2awQAvD_BwE&gclsrc=aw.ds and https://www.abcam.com/products?keywords=JAML&selected.productType=Primary+antibodies &selected.targetName=JAML (each last accessed on Oct. 31, 2022)

In another aspect, the checkpoint inhibitor of the combination or composition comprises GS4224, AMP-224, CA-327, CA-170, BMS-1001, BMS-1166, peptide-57, M7824, MGD013, CX-072, UNP-12, NP-12, or a combination of two or more thereof.

In one aspect of the combination or composition, the checkpoint inhibitor comprises one or more selected from an anti-PD-1 agent, an anti-PD-L1 agent, an anti-CTLA-4 agent, an anti-LAG-3 agent, an anti-TIM-3 agent, an anti-TIGIT agent, an anti-VISTA agent, an anti-B7-H3 agent, an anti-BTLA agent, an anti-ICOS agent, an anti-GITR agent, an anti-4-1BB agent, an anti-OX40 agent, an anti-CD27 agent, an anti-CD28 agent, an anti-CD40 agent, and an anti-Siglec-15 agent. In another aspect, the checkpoint inhibitor comprises an anti-PD1 agent or an anti-PD-L1 agent. In a further aspect, the anti-PD1 agent comprises an anti-PD1 antibody or an antigen binding fragment thereof. Non-limiting examples of an anti-PD1 antibody comprises nivolumab, pembrolizumab, cemiplimab, spartalizumab, camrelizumab, sintilimab, tislelizumab, toripalimab, AMF 514, or a combination of two or more thereof. In one embodiment, the anti-PD-L1 agent comprises an anti-PD-L1 antibody or an antigen binding fragment thereof. In another aspect, the anti-PD-L1 antibody comprises avelumab, durvalumab, atezolizumab, envafolimab, or a combination of two or more thereof. In another embodiment, the checkpoint inhibitor comprises an anti-CTLA-4 agent. In one aspect, the anti-CTLA-4 agent comprises an anti-CTLA-4 antibody or an antigen binding fragment thereof. In a further embodiment, the anti-CTLA-4 antibody comprises ipilimumab, tremelimumab, zalifrelimab, or AGEN1181, or a combination thereof. In one embodiment, the checkpoint inhibitor comprises an anti-PD1 agent or an anti-PD-L1 agent and an anti-CTLA-4 agent. In another embodiment, the anti-PD1 agent comprises an anti-PD1 antibody or an antigen binding fragment thereof. Non-limiting examples of the anti-PD1 antibody comprises nivolumab, pembrolizumab, cemiplimab, spartalizumab, camrelizumab, sintilimab, tislelizumab, toripalimab, AMF 514, or a combination of two or more thereof. Alternatively, the anti-PD-L1 agent comprises an anti-PD-L1 antibody or an antigen binding fragment thereof, and non-limiting examples thereof include avelumab, durvalumab, atezolizumab, envafolimab, or a combination of two or more thereof. In another aspect, the anti-CTLA-4 agent comprises an anti-CTLA-4 antibody or an antigen binding fragment thereof, and non-limiting examples of such include ipilimumab, tremelimumab, zalifrelimab, or AGEN1181, or a combination thereof.

### EXPERIMENTAL

### Materials and Methods

Mice. C57BL/6J (stock no. 000664), OT-I (stock no. 003831). CD45.1 (stock no. 002014) and CD8^{-/-} (stock no. 002665) mice were obtained from The Jackson Laboratory. In all experiments, female mice (6-12 weeks old) were used. In the vivarium, housing temperature was kept within the range of 20-24° C.; humidity was monitored but not controlled and ranged from 30 to 70%. The mice were kept in 12 h light-dark cycles (06:00-18:00 light). The La Jolla institute for Immunology Animal Ethics Committee approved all animal work.

Tumor cell lines. MC38-OVA cells, a gift from the S. Fuchs laboratory (UPenn) were approved for use by M. Smyth (Peter MacCallum Cancer Center). The B16F10-OVA cells were a gift from the J. Linden laboratory (LJI). All cell lines tested negative for mycoplasma infection and were subsequently treated with Plasmocin (InvivoGen) to prevent contamination.

Tumor models. Tumor models were used as described before⁷. The mice were s.c. inoculated with 2×10⁶ MC38-OVA cells (CXADR^{+/+} or CXADR^{-/-}) or 1-1.5×10⁵ B16F10-OVA cells into the right flank. The mice were injected intraperitoneally at indicated time points with either 200 μg isotype control antibodies, anti-PD-1 (29F1. A1, Bioxcell) or anti-JAML (4E10, Biolegend). Tumor size was monitored every 2-3 days to ensure that the tumors did not exceed 25 mm in diameter. At experimental endpoint, tumors were harvested and tumor-infiltrating lymphocytes were analyzed. Tumor volume was calculated as described previously⁷.

In vitro assays. CD8⁺ T cells were labeled with CellTrace Violet (ThermoFisher). Subsequently, 20,000 cells were added to 96-well cell-culture plates containing 40,000 CXADR^{+/+} or CXADR^{-/-} cells respectively in 200 μl complete RPMI medium. CD8⁺ T cell proliferation was determined three days later.

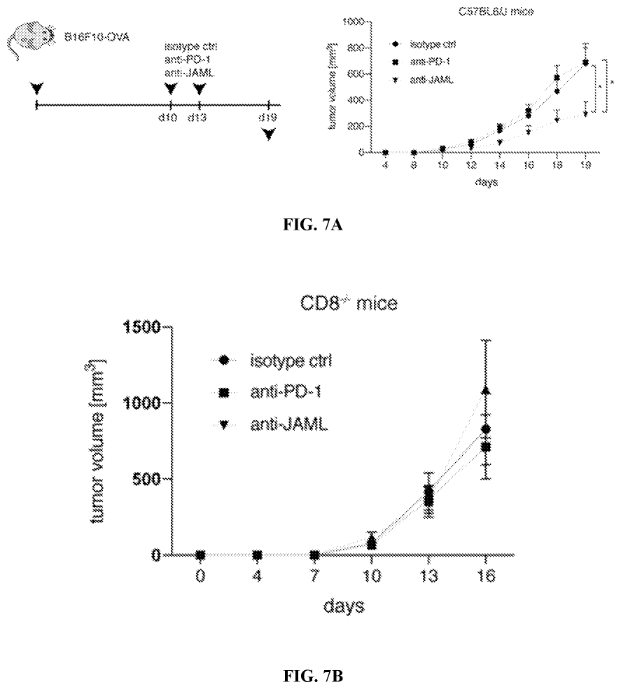

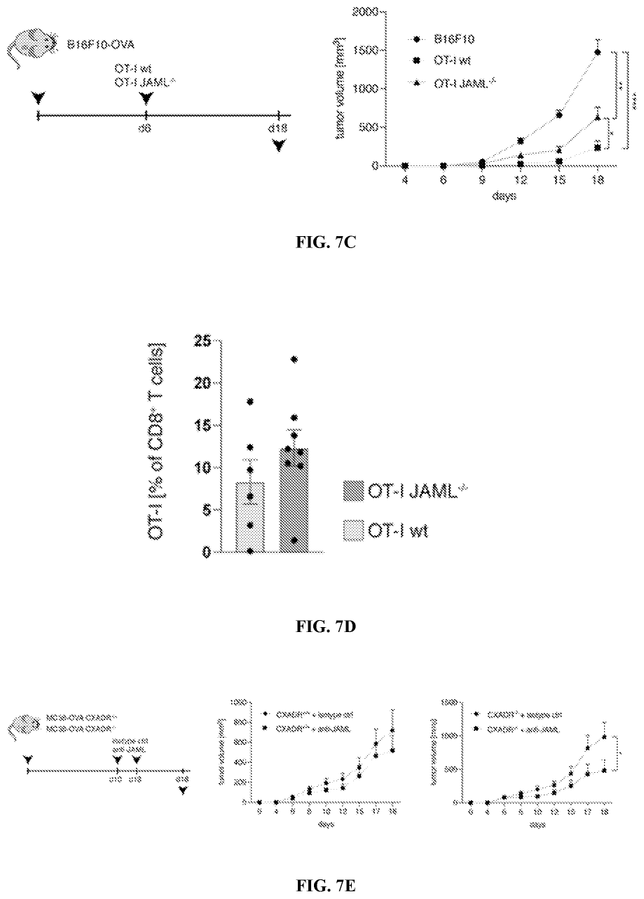

Flow cytometry. Lymphocytes were isolated from the liver or spleen by mechanically dispersing the cells through a 70 m cell strainer (Miltenyi) generating single-cell suspensions. RBC lysis (BioLegend) was performed to lyse and remove red blood cells. Tumors were harvested and TILs were isolated by dispersing the tumors in 2 ml sterile PBS and subsequently incubating the samples at 37° C. with liberase DL (Roche) and DNase I (Sigma) for 15 min. Colonic tissue cell were isolated as described previously³⁸. To create single-cell suspensions, the samples (tumor, lover, colon or spleen) were passed through a 70-μm cell strainer. The cells were kept in staining buffer (PBS with 2 mM EDTA and 2% FBS), FcγR blocked (clone 2.4G2, BD Biosciences), followed by staining with the indicated antibodies at 4° C. for 30 min; secondary stains were conducted where indicated for selected markers. The samples were then either sorted or fixed and stained intracellularly with a FOXP3 transcription factor kit (eBioscience) according to the manufacturer's instructions. To determine cell viability, fixable viability dye (ThermoFisher) was used in all staining reactions. For the bulk RNA-seq analyses, Applicants sorted tumor-infiltrating T_{REG} or CD8⁺ cells based on the expression of the indicated markers (FIG. 7A). All samples were sorted on a BD FACS Fusion system or acquired on a BD FACS Fortessa system (both BD Biosciences) and then analyzed using FlowJo 10.4.1.

Histology and immunohistochemistry. The primary antibodies used for immunohistochemistry included anti-CD8 (pre-diluted; C8/144B, Agilent Dako), anti-JAML (1:100; Atlas, HPA047929), anti-CD103 (1:500; Abcam, abl29202), CK (1:5; Dako, AE1/AE3) The samples for the immunohistochemical analyses were prepared, stained and analyzed as previously described⁷. Cells were identified by nucleus detection and cytoplasmic regions were simulated up to 5 μm per cell; protein expression was measured using the mean staining intensity within the simulated cell regions.

Bulk RNA-seq. Total RNA was purified from murine tumor-infiltrating TREG (LIN⁻CD45⁺CD3⁺CD4⁺FOXP3⁺) and CD8⁺ T (LIN⁻CD45⁺CD3⁺CD8⁺) cells using the miRNeasy kit (Qiagen) and quantified as previously described^{9,39}. RNA-seq libraries were prepared with a Smart-seq2 protocol and were sequenced on an Illumina platform⁴⁰. Quality-control was applied as previously described⁹ and data were analyzed as described previously⁷.

Meta-analysis of published single-cell RNA-seq studies. The meta-analysis was conducted as described previously⁷. In brief, nine published single-cell RNA-seq datasets^{28,41-48} of CD4-expressing and CD8-expressing (n=22,410) tumor-infiltrating T cells were integrated with UMAP using the R package Seurat v3.0. For each dataset, cells that expressed fewer than 200 genes were considered outliers and discarded. Applicants integrated data from all cohorts using the alignment by the ‘anchors’ option in Seurat 3.0 as described previously⁷. Briefly, the alignment is a computational strategy to ‘anchor’ diverse datasets together, facilitating the integration and comparison of single-cell measurements from different technologies and modalities. The ‘anchors’ correspond to similar biological states between datasets. These pairwise correspondences between datasets allows the transformation of datasets into a shared space regardless of the existence of large technical and/or biological divergences. This improved function in Seurat 3.0 allows integration of multiple RNA-seq datasets generated by different platforms⁴⁹. Applicants used the FindIntegrationAnchors function to find correspondences across the different study datasets with default parameters (dimensionality=1:30). Furthermore, Applicants used the IntegrateData function to generate a Seurat Object with an integrated and batch-corrected expression matrix. In total, 22,410 cells and 2,000 most variable genes were used for clustering. Applicants used the standard workflow from Seurat, scaling the integrated data, finding relevant components with principal-component analysis and visualizing the results with UMAP. The number of relevant components was determined from an elbow plot. UMAP dimensionality reduction and clustering were applied with the following parameters: 2,000 genes; 30 principal components; resolution, 0.4. The cells that were used for the integration were selected from clusters labeled in the original studies as tumor CD4+ T cells and from pretreatment samples when necessary.

Single-cell transcriptome analysis. Murine CD45⁺JAML⁺ cells from three B16F10-OVA tumor-bearing mice were isolated and prepared as described above. Cells from each mouse were barcoded with murine Totalseq-B antibodies. Cells were sorted and complementary DNA libraries were constructed using the standard 10× Genomics sequencing protocol. The antibody capture data were analyzed using custom scripts (github.com/vijaybioinfo/ab_capture), as previously described⁷. n=8,474 cells were sequenced and cells with fewer than 1,500 and more than 6,000 expressed genes, less than 1,000 and more than 50,000 counts, and more than 5% mitochondrial counts were filtered out. 5,976 cells were used for downstream analyses. For clustering with Seurat (3.0), Applicants used 15 principal components from a set of highly variable genes (n=609) taking 20% of the variance after filtering out genes with a mean expression of less than 0.01. Differential gene expression was calculated using MAST (P<0.05, log 2FC>0.25) as described previously⁷.

Quantification and statistical analysis. The number of mice per group and statistical tests used can be found in the figure legends. Details on sample elimination, quality control, and displayed data are stated in the figure legends and methods. Sample sizes were based on previous experiments and published studies to ensure reliable statistical testing accounting for variability between groups. Mice that did not develop tumors by 10 days after inoculation, before therapeutic intervention, were not included in the analyses. Low quality samples were excluded from the analyses and stated in the methods section. Data in heatmaps are displayed as log 2-normalized z-scores. Experiments were reproduced in at least two independent experiments. Age and sex-matched mice were used in the experiments and animals were randomly assigned to the experimental groups. Statistical analyses were performed using GraphPad Prism 9.H3K27ac ChIP-seq and HiChIP data for 6 common immune cell and ATAC-seq data for 15 DICE cell types have been previously reported^{24,25,50} and are available from the database of Genotypes and Phenotypes (dbGaP; accession number: phs001703.v4.pl). ATAC-seq data for TRM cells and non-TRM cells were previously reported¹⁰ and are available on GEO (accession number: GSE111898). NFATC1 and NFATC2 ChIP-seq data was obtained from GEO (accession number: GSM2810039 and GSM2810040 respectively).

### Results

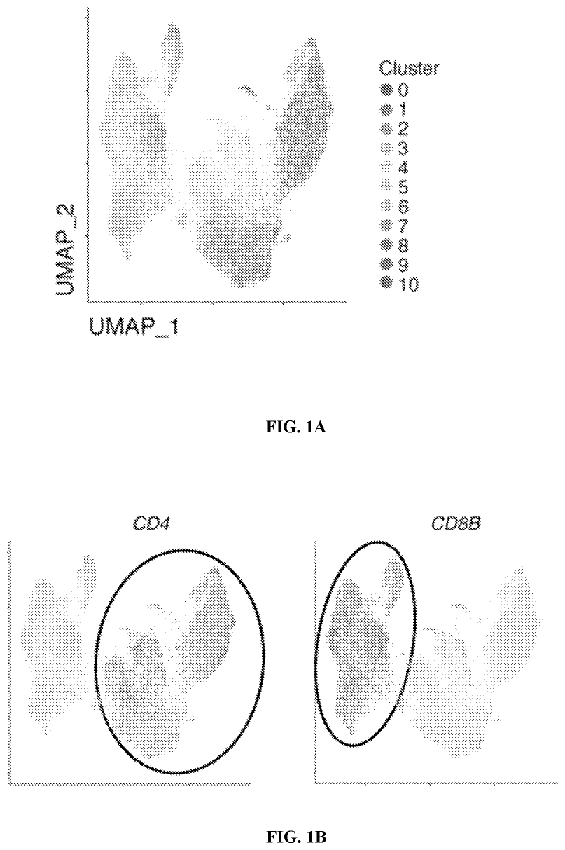

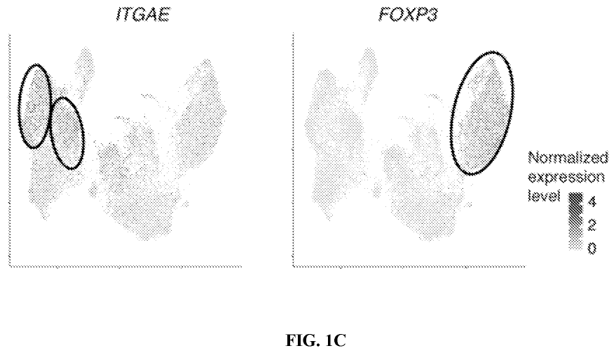

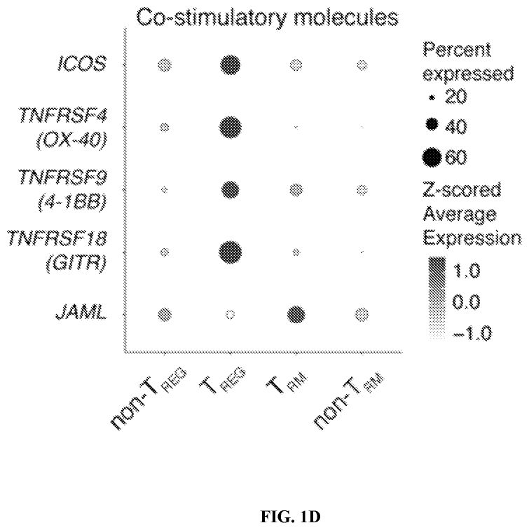

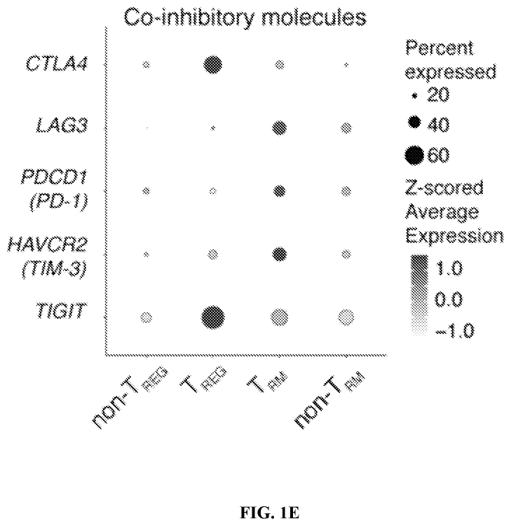

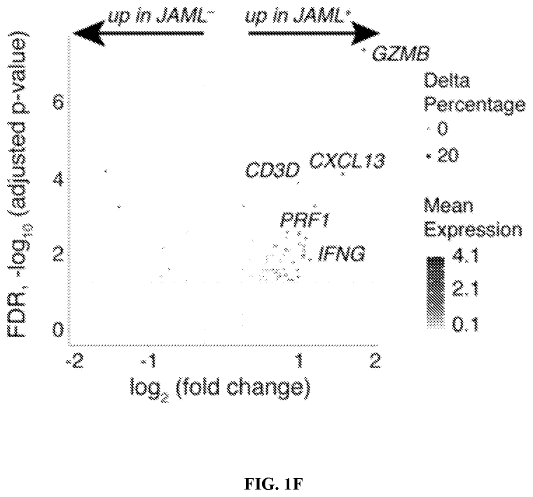

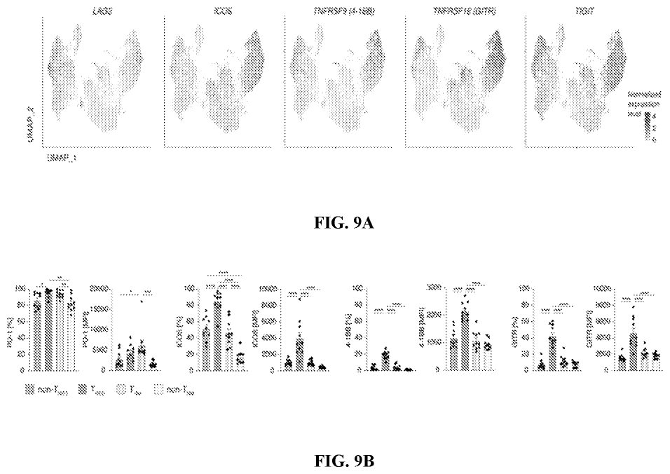

JAML is enriched in tumor-infiltrating CD8⁺ T_{RM} cells of multiple cancer types. As few studies have thoroughly assessed the level and breadth of immunotherapy target expression in T cell subsets in the TME. Applicants integrated and analyzed 9 published single-cell RNA-seq datasets of tumor-infiltrating CD4⁺ and CD8⁺ T cells (n=22,410 cells) spanning 7 different cancer types. Data visualization using uniform manifold approximation and projection (UMAP) revealed 10 distinct T cell subsets (FIG. 1A-C) that differed substantially in their expression of several co-stimulatory and co-inhibitory receptors (FIG. 9A). Given the opposing roles of CD4⁺FOXP3⁺ (T_{REG}) cells and CD8⁺ T_{RM} cells in anti-tumor immunity and immunotherapy efficacy (refs.^{7,9,12,19-23}), Applicants assessed the transcript expression levels of several immunotherapy targets in these major CD4⁺ (non-T_{REG} and T_{REG}) and CD8⁺ (T_{RM} and non-T_{RM}) T cell subsets (FIG. 1B-D). Importantly, T_{REG} cells, when compared to the other T cell subsets, expressed higher levels of transcripts encoding for several co-stimulatory and co-inhibitory immunotherapy drug targets currently in clinical use or clinical trials (e.g., 4-1BB, ICOS, OX-40, GITR, TIGIT) (FIG. 1D, E), while some co-inhibitory receptors were expressed on all assessed T cell subsets (FIG. 1E). On the other hand, Applicants found JAML transcripts to be expressed at relatively higher levels by CD8⁺ T_{RM} cells when compared to T_{REG} cells (FIG. 1D). Given that this expression pattern was observed in many cancer types, Applicants next verified immunotherapy target protein expression levels on tumor-infiltrating T_{REG} and CD8⁺ T cells in patients with early stage and treatment naïve non-small cell lung cancer (NSCLC) (FIG. 9B). These analyses corroborated Applicants' findings from single-cell RNA-seq data and confirmed that several immunotherapy target molecules are expressed at higher levels by tumor-infiltrating T_{REG} cells.

Because co-stimulatory molecules enhance TCR-dependent cell activation, proliferation, and effector functions, Applicants next tested whether JAML-expressing T cells exhibit transcriptional features of superior functionality when compared to their JAML-non-expressing counterparts. Notably, Applicants found that JAML-expressing T_{RM} cells expressed higher levels of transcripts encoding for cytotoxicity molecules (Granzyme B, Perforin) and effector cytokines (IFN-γ, CXCL13) when compared to T_{RM} cells not expressing JAML (FIG. 1F), suggesting that JAML expression marks T_{RM} cells with enhanced functional properties, or that JAML itself enhances functionality. Together, these data suggest that immune suppressive T_{REG} cells can get activated by agonistic antibodies targeting co-stimulatory molecules or by antibodies blocking co-inhibitory molecules, thus dampening their treatment efficacy. Conversely, JAML is primarily expressed by CD8⁺ T_{RM} cells, implying that agonistic anti-JAML antibodies would preferentially activate CD8⁺ T_{RM} cells with superior functionality and thus augment anti-tumor immunity.

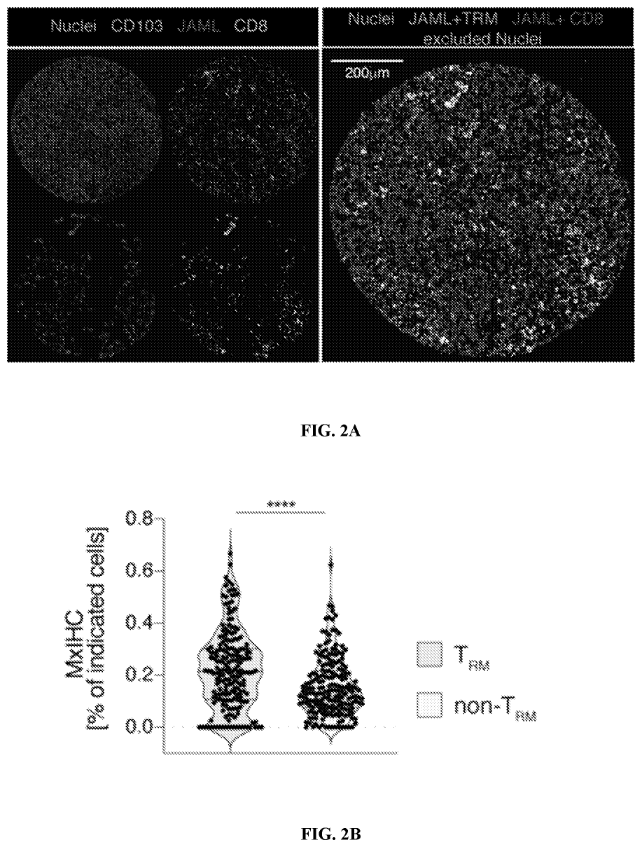

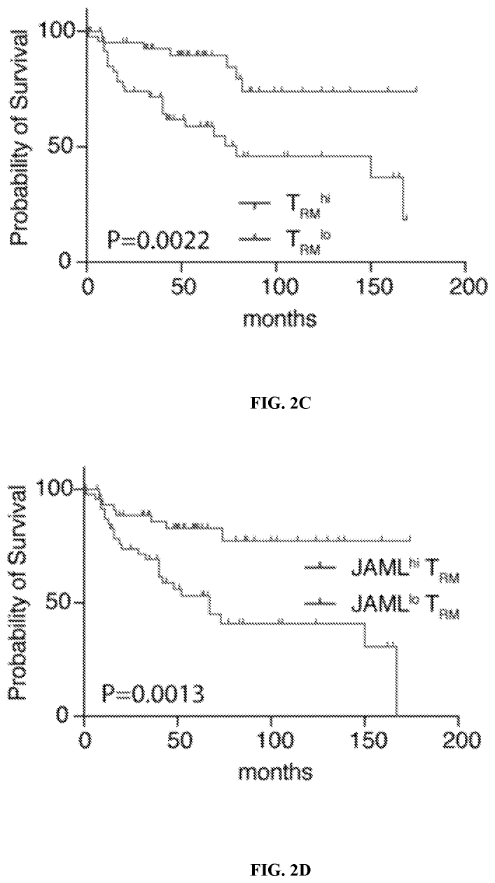

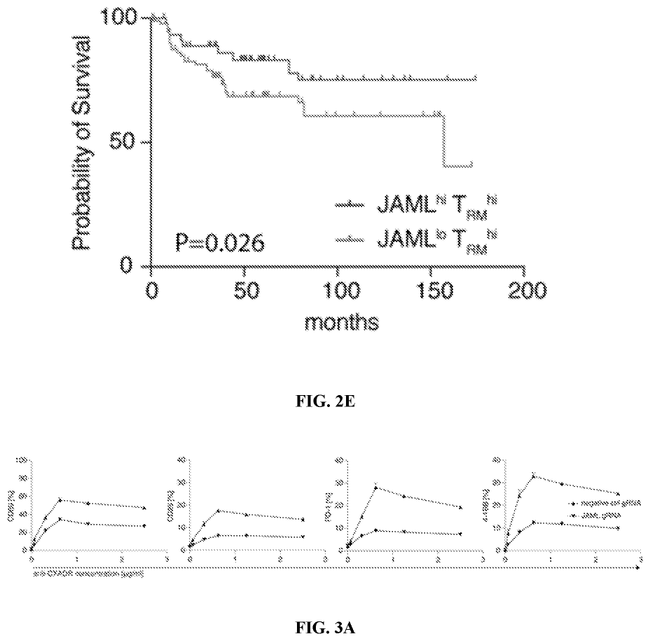

JAML expression on TR_{M} cells is associated with improved survival outcomes. Based on these data and given that Applicants found that JAML is primarily expressed on highly functional T_{RM} cells in tumor tissues¹⁰, Applicants reasoned that expression of JAML in T_{RM} cells may positively influence their anti-tumor activity and thus survival outcomes, and examined such association in a large cohort of patients with head and neck squamous cell carcinoma (HNSCC) (n=194). As expected, Applicants found that a greater proportion of CD8⁺ T_{RM} cells expressed JAML when compared to CD8⁺ non-T_{RM} cells (FIG. 2A, 2B). Consistent with previous reports in NSCLC⁹ or early-stage triple negative breast cancer¹¹, Applicants demonstrate that intratumoral density of CD8⁺ T_{RM} cells is significantly associated with improved patient survival (FIG. 2C). Moreover, HNSCC patients with higher proportions of JAML-expressing CD8₊ T_{RM} cells in the tumor had significantly better long-term overall survival outcomes when compared to those with lower proportions of JAML-expressing T_{RM} cells (JAML^{low} T_{RM} tumors) (FIG. 2D). This beneficial effect on survival outcomes was maintained even when analysis was restricted to patients with a high density of T_{RM} cells in tumors (FIG. 2E), an immune profile that has been shown to independently influence survival outcomes (FIG. 2C). These results suggest that expression of JAML is likely to confer T_{RM} cells with enhanced anti-tumor activity.

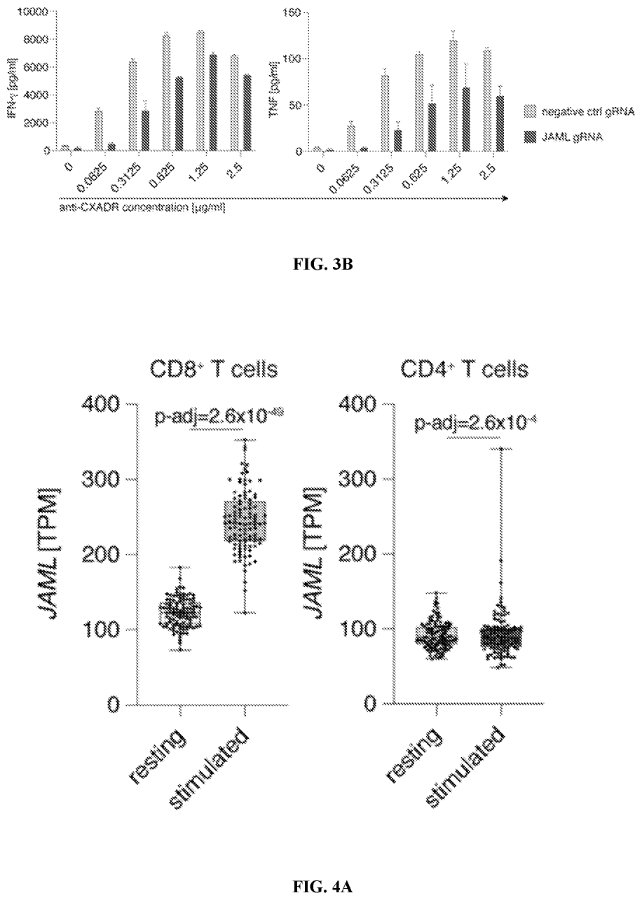

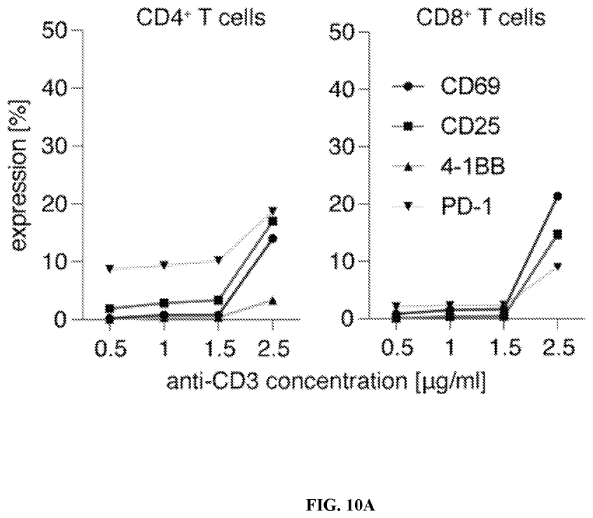

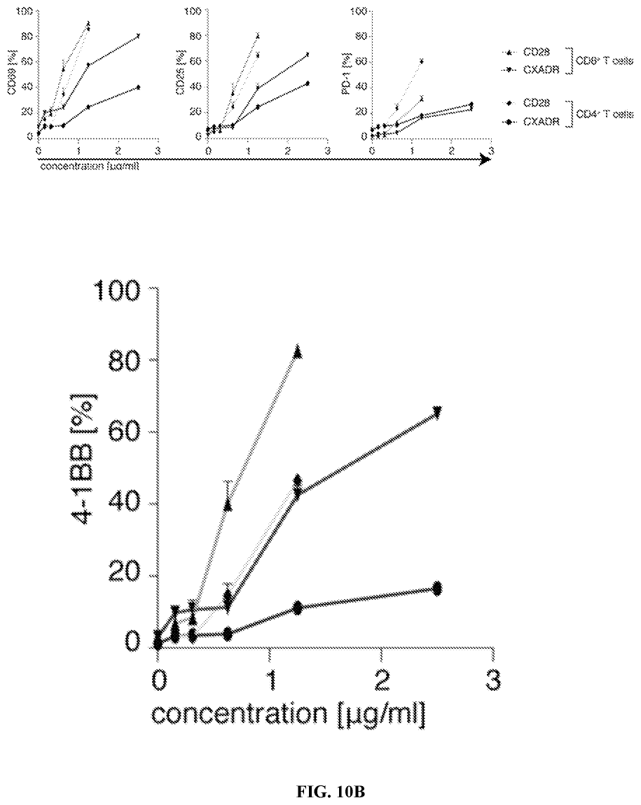

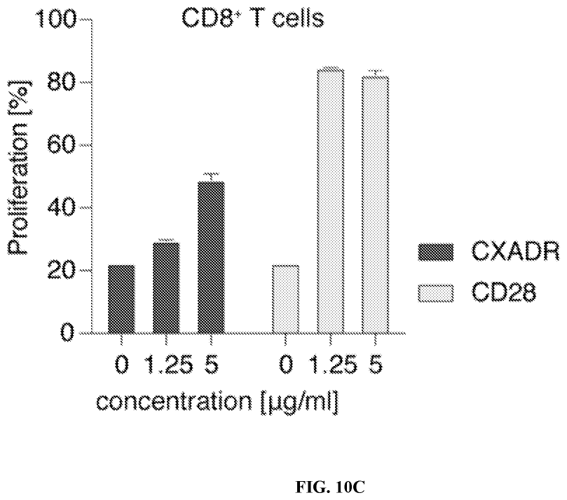

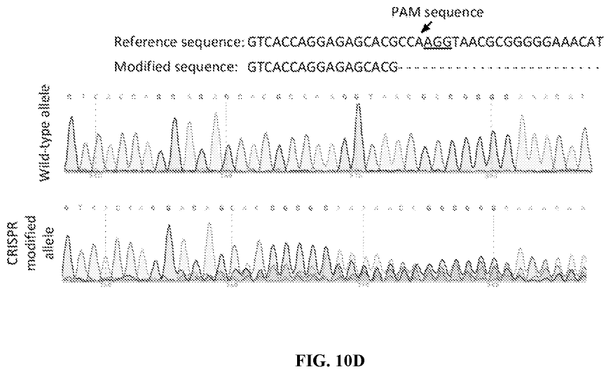

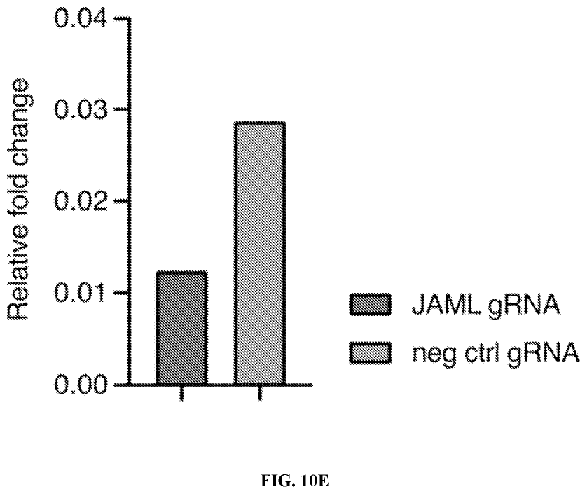

JAML functions as a co-stimulatory signal in human αβ T cells. As previous studies implied that JAML might not function as a co-stimulatory molecule in αβ T cells^{16,17}, Applicants tested whether ligand binding to JAML triggers activation of human CD4⁺ and CD8⁺ T cells. At a sub-optimal concentration (0.5 g/ml) of anti-CD3, which by itself did not induce cell activation (FIG. 10A), JAML ligation by its endogenous ligand CXADR led to rapid and dose-dependent upregulation of the early activation markers CD69, CD25, PD-1 and 4-1BB (FIG. 10B) and cell proliferation (FIG. 10C). Even at low concentrations, Applicants found that JAML, like the co-stimulatory molecule CD28, potently activated CD4⁺ and CD8⁺ T cells (FIG. 10B). To confirm that CXADR activates T cells through ligation of JAML, Applicants knocked down JAML expression in primary human CD8⁺ T cells utilizing Crispr-Cas9 and assessed co-stimulatory effects of CXADR. Transfection of CD8⁺ T cells with a JAML guide RNA altered the nucleotide sequence in the targeted JAML gene region (Exon 2), presumably driven by CRISPR-Cas9-mediated insertion or deletion events (FIG. 10D), significantly diminished JAML expression (FIG. 10E) and reduced T cell activation and cytokine secretion by CXADR co-stimulation (FIG. 3A,B). Contrary to the previous report¹⁷, these data demonstrate that JAML facilitates potent co-simulation in αβ T cells.

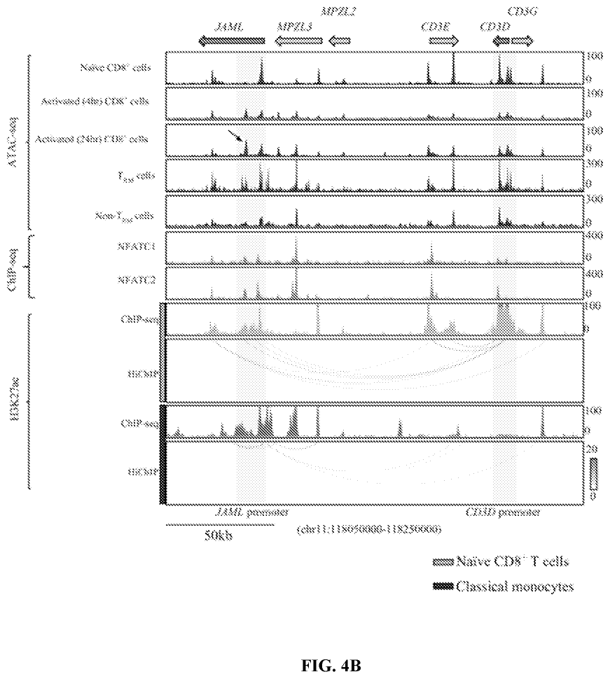

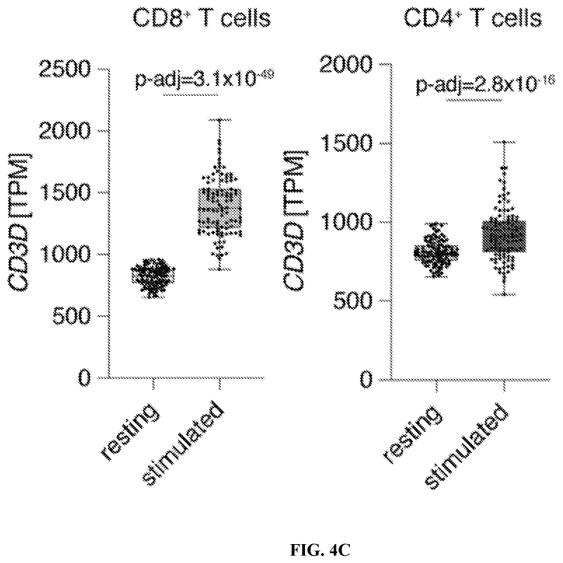

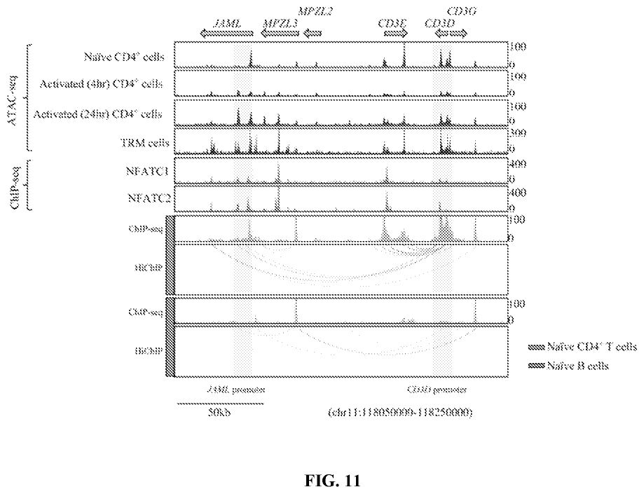

JAML expression is regulated by interactions between the CD3D and JAML promoters. Utilizing previously published dataset²⁴, Applicants found that TCR stimulation more significantly increased JAML expression in human CD8⁺ T cells compared to CD4⁺ T cells (log 2 fold change 1.24 versus 0.37 in CD8⁺ and CD4⁺ T cells, respectively; FIG. 4A). To investigate how TCR signaling induces JAML expression in αβ T cells, Applicants first examined transposase accessible regions (ATAC-seq peaks) in the JAML locus in resting and stimulated human CD8⁺ and CD4⁺ T cells (FIG. 4B and FIG. 11A). Activation induced a strong ATAC-seq peak in the JAML intronic region (FIG. 4B) that also contained binding sites for NFAT, a key transcription factor involved in activation of genes following TCR activation.

Notably, human tumor-infiltrating T_{RM} cells displayed greater accessibility at the JAML promoter and the pertaining activation-induced intronic ATAC-seq peak region when compared to non-T_{RM} cells. Applicants also found several NFAT binding sites in the promoter regions of upstream genes like CD3D and CD3G which encode for key components of the TCR, and which like JAML, showed increased expression following activation (FIG. 4B). Importantly, by examining the 3D chromatin interaction map of the extended JAML locus in primary human T cells²⁵, Applicants found that the JAML promoter and the activation-induced intronic cis-regulatory region strongly interacted with the neighboring CD3D promoter region (FIG. 4B), suggesting that they are likely to be involved in regulating JAML expression. Accordingly, Applicants found minimal interactions between these gene loci in other immune cell types (i.e., B cells or monocytes) that lack active CD3D promoter regions, indicative of a T cell-specific cis-regulatory control of JAML expression (FIG. 4B and FIG. 11A). As Applicants have previously demonstrated that promoter-promoter interactions play a major role in regulating gene expression²⁵, Applicants' data imply that the respective promoter regions of CD3D on the one hand, and JAML on the other hand, may act as reciprocal enhancers inducing each other's expression. TCR signaling is likely to increase NFAT binding and thus the transcriptional activity of the CD3D promoter, thus driving its own expression (FIG. 4C) and with it, the expression of JAML through long-range cis-regulatory interactions. Together, these data demonstrate how and why JAML expression is induced in human T cells by TCR engagement and implies a T cell-specific inducible expression profile of this co-stimulatory molecule. Crucially and in line with Applicants' previous study¹⁰, these findings suggest that JAML expression might also be enriched in highly functional antigen-specific CD8⁺ T_{RM} cells (i.e., reactive to tumor associated-antigens or neoantigens) driven by TCR-specific antigen-recognition and subsequent upregulation of JAML expression.

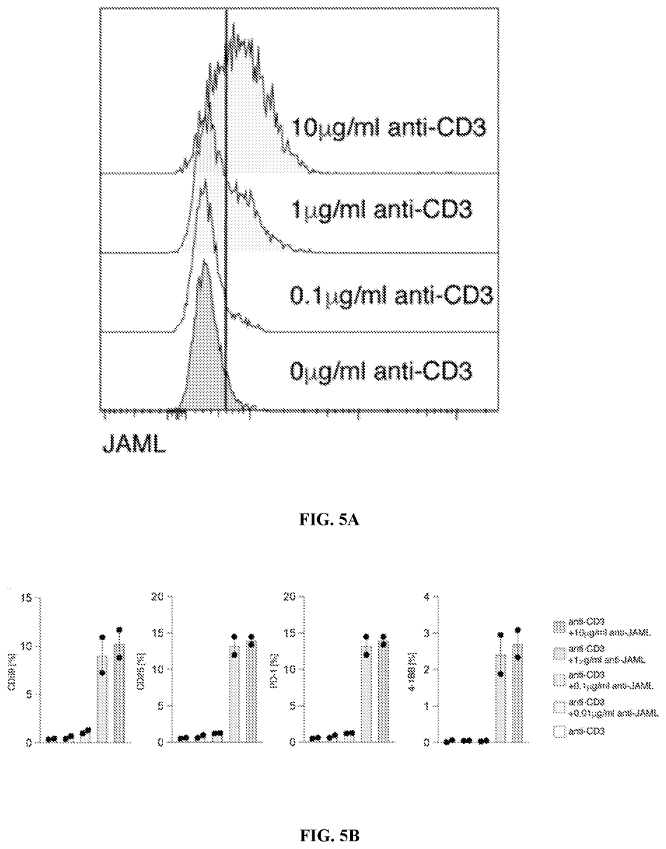

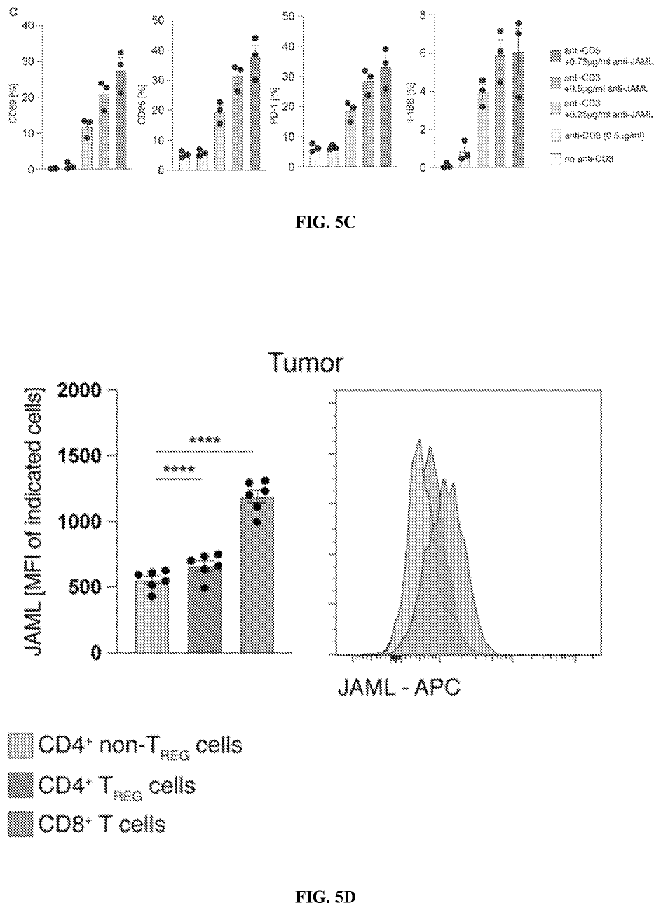

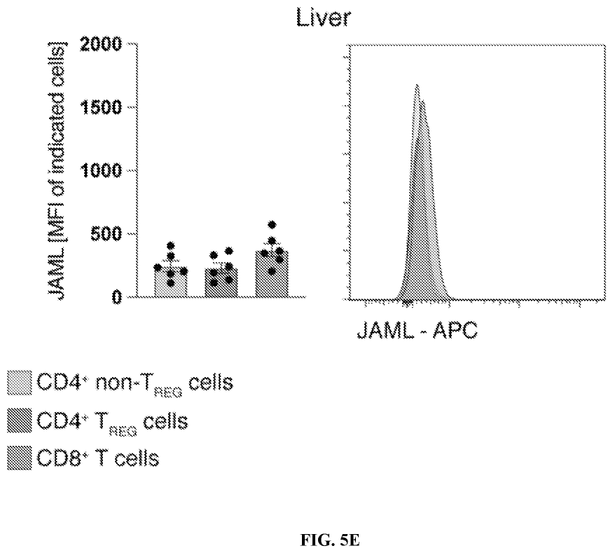

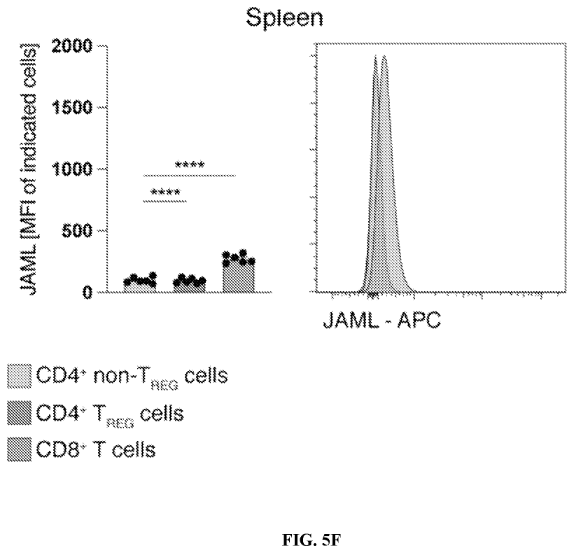

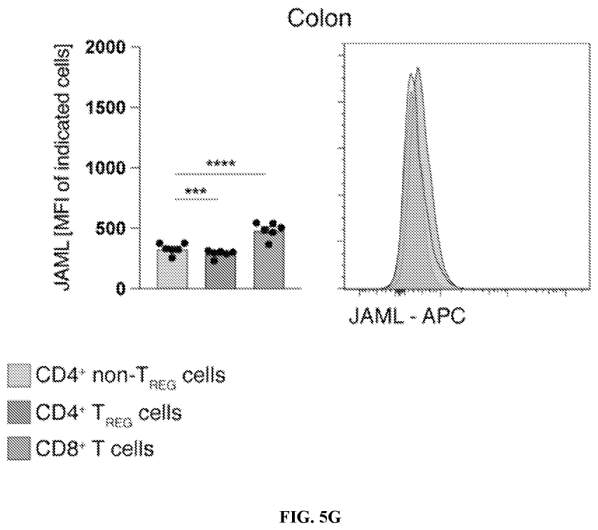

Murine CD8⁺ TILs selectively express high levels of JAML. Applicants next assessed whether Applicants' findings in human T cells are also applicable to murine T cells. Similar to human αβ T cells, Applicants found that TCR-signaling rapidly upregulated JAML expression on murine αβ CD8⁺ T cells (FIG. 5A). Upon co-stimulation with an agonistic anti-JAML antibody¹⁷ and anti-CD3, Applicants observed a substantial upregulation of surface activation markers, confirming that JAML can function as a co-stimulatory molecule even in murine αβ T cells, a finding replicated in a recent study¹⁸ (FIG. 5B and FIG. 11A, FIG. 11B). Importantly, stronger TCR stimulation resulted in higher expression of JAML and thus required a ˜10-fold lower concentration of agonistic anti-JAML antibody to activate CD8+ T cells even more strongly (FIG. 5C), implying that high avidity T cells (i.e. tumor antigen-reactive CD8+ T cells) expressing high levels of JAML, are highly sensitive to agonistic anti-JAML antibody treatment, even at relatively low concentrations. These data moreover imply that bi-specific T cell engagers (BiTEs) binding JAML and tumor-associated antigens or neoantigens might preferentially activate tumor antigen-specific CD8+ T cells (i.e. TRM cells and stem-like cells), as those would express the highest levels of JAML. Due to the nature of JAML expression (upregulated upon TCR engagement), monovalent binding, as observed on BiTEs or bi-specific antibodies, would confer high specificity, as it would primarily activate such JAMLhi-expressing T cells in the TME. Thus, unlike BiTEs or bispecifics which bind CD28 on T cells, a co-stimulatory molecule which is ubiquitously expressed on most T cells, JAML-binding antibodies might elicit similar efficacy with significantly reduced toxicity, as they would not activate bystander cells. These results supported the rationale for testing the co-stimulatory function of JAML in vivo, especially in the context of tumor models, to determine if JAML could be utilized as a cancer immunotherapy target that potentially surpasses treatment efficacy of current immunotherapy drugs due to its low expression on immunosuppressive T_{REG} cells. Applicants tested this hypothesis in a murine melanoma model that is refractory to anti-PD-1 therapy. Given that short-term s.c. syngeneic tumor models do not induce robust anti-tumor CD8⁺ T_{RM} responses²³, Applicants first assessed JAML expression levels on CD4⁺ (T_{REG} and non-T_{REG}) and CD8⁺ TILs of B16F10-OVA tumor-bearing mice. Consistent with Applicants observations in human TILs, JAML was expressed at significantly higher levels in tumor-infiltrating CD8⁺ T cells when compared to tumor-infiltrating T_{REG} cells and CD4⁺ non-T_{REG} cells (FIG. 5C), implying that treatment with agonistic JAML antibodies should preferentially activate CD8⁺ T cells over immunosuppressive T_{REG} cells and thus enhance anti-tumor immune responses. Importantly, Applicants found relatively low expression of JAML in CD4⁺ and CD8⁺ T cells present in spleen, colon and liver of tumor-bearing mice (FIG. 5D-F), suggesting that therapies activating JAML are likely to act primarily on CD8⁺ T cells within the tumor microenvironment (TME) and might therefore exert a favorable safety profile by not engaging T cells at common sites of immune-related toxicity.

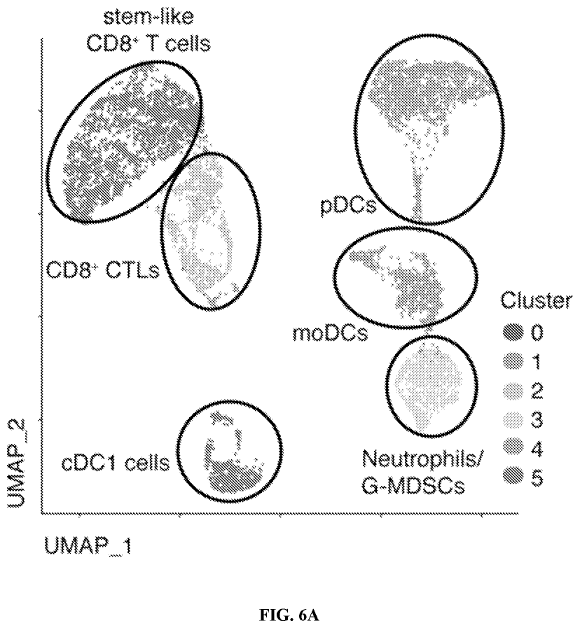

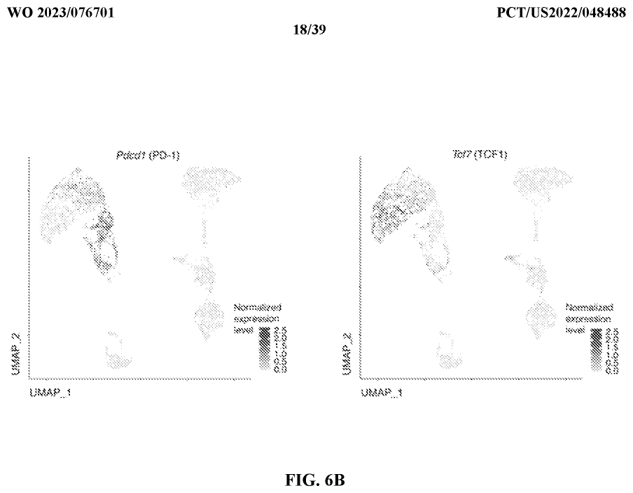

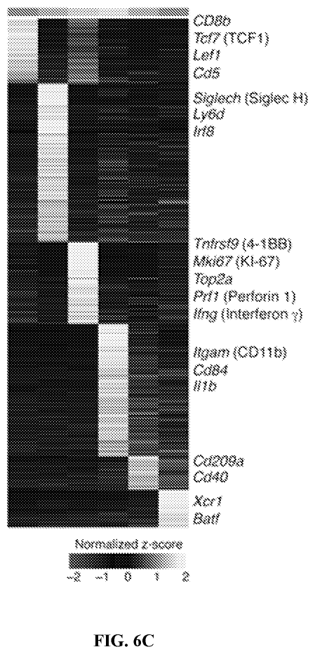

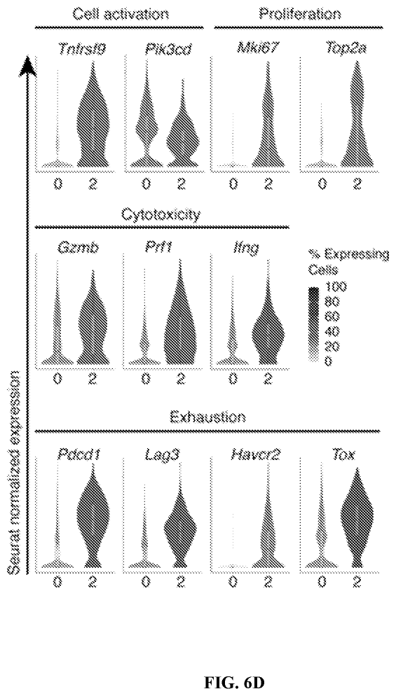

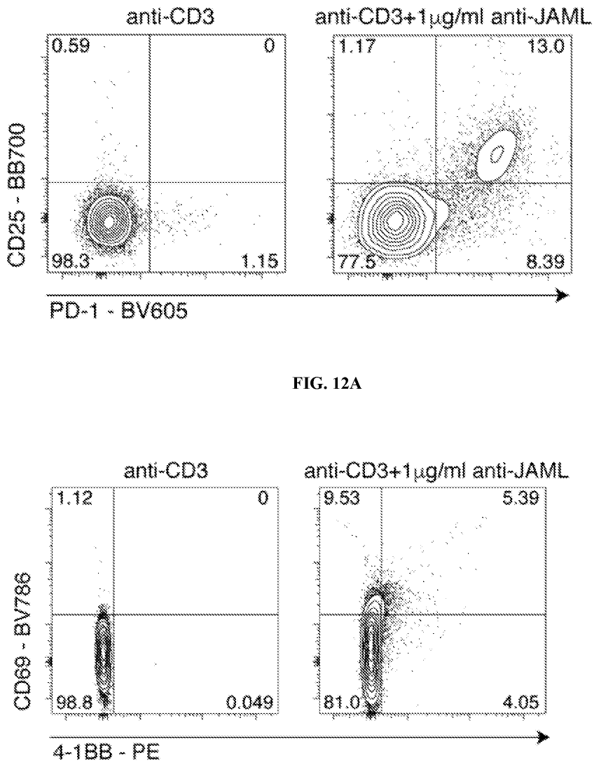

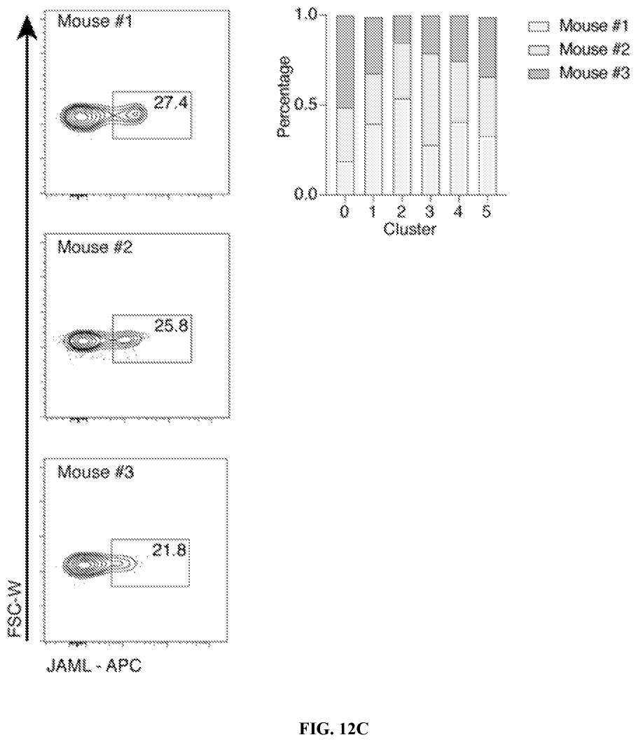

‘Stem-like’ CD8⁺ TILs express JAM L. To determine the properties of tumor-infiltrating CD8⁺ T cell subsets that express JAML and to explore the other immune cell types that express JAML in an unbiased manner, Applicants performed single-cell RNA-sequencing of JAML-expressing CD45⁺ cells present in primary late-stage tumor tissue of 3 individual B16F10-OVA tumor-bearing mice (FIG. 12C). Unbiased clustering depicted by UMAP analysis revealed 6 clusters, and importantly, substantiated that JAML expression in the T cell compartment is restricted to CD8⁺ TILs (cluster 0,2; FIG. 6A). The JAML-expressing CD8⁺ TILs clustered into two distinct subsets that displayed striking differences in the expression of transcripts encoding for TCF1 (Tcf7) and PD-1 (Pdcd1) (FIG. 6B). The Pdcd1-low cluster (cluster 0) expressed high levels of molecules linked to ‘stem-like’ properties (i.e., Tcf7, Lef1, Cd5), which have been shown to be important for sustaining anti-viral and anti-tumor immune responses²⁶⁻²⁸ (FIG. 6C, 6D). In contrast, the Pdcd1-enriched CD8⁺ T cell cluster (cluster 2), when compared to cluster 0 CD8⁺ T cells, displayed significantly higher expression of several transcripts linked to T cell activation (Tnfrsf9, Pik3cd), cytotoxicity (Gzmb, Prf1, Ifng) and cell proliferation (Mki67, Top2a), which suggested recent TCR activation by antigen-encounter, presumably directed to tumor antigens. The Pdcd1-enriched cluster also expressed high levels of other transcripts linked to exhaustion (Lag3, Havcr2, Tox) (FIGS. 6B-6D), and in agreement with previous studies^{29,30}, these results indicate that expression of these exhaustion-like markers in murine TILs is unlikely to impede their functionality. Instead, it appears that expression of these molecules is a necessary adaptation to survive in the TME³¹ and to potentially limit immunopathology³², and likely marks effector T cells potentially responding to tumor antigens. Thus, these CD8⁺ T cells by virtue of co-expressing both PD-1 and JAML are likely to be activated by both agonistic anti-JAML antibodies and anti-PD1 therapies, and, importantly, when these therapies are combined, synergistic activation and enhanced anti-tumor responses are likely. Whereas, JAML-expressing Pdcd1-low cluster, which comprises of stem-like cells, is likely to be preferentially activated by agonistic anti-JAML antibodies when compared to anti-PD1 therapies. Applicants corroborated these data on the protein level, demonstrating that few CD8+ T cells co-expressed PD-1 and TCF1, implying that anti-PD-1 treatment does not activate stem-like CD8+ T cells. Conversely, Applicants found similar ratios of JAML-expressing CD8+ T expressing PD-1 or TCF1 (FIG. 6E), implying that anti-JAML treatment might induce a sustained anti-tumor immune response as it would activate both stem-like and effector CD8+ T cells.

Although, other lymphocyte subsets like T_{REG} were not represented in JAML-expressing immune cells, Applicants found several dendritic cell subsets expressed JAML (clusters 1,4,5) and corroborated previous reports of JAML-expressing neutrophils or granulocyte-derived myeloid-derived suppressor cells MDSCs (cluster 3)^{33,34} (FIG. 6A, C) in the TME. This result suggests that therapies targeting JAML may have ‘on-target/off-cell’ effects on JAML-expressing myeloid and dendritic cell compartments, but whether JAML agonistic antibodies modulate the functional activity of such cells remains to be explored. Conversely, anti-JAML antibodies are unlikely to elicit ‘on-target/off-tumor’ effects, as T cells in other non-malignant organs lack substantial expression of JAML, and are thus unlikely to cause end-organ toxicity.

The anti-tumor effects of anti-JAML depend on CD8⁺ TILs. In line with previous studies³⁵, Applicants found no changes in tumor volume upon anti-PD-1 monotherapy in the B16F10-OVA tumor model (FIG. 7A). Conversely, treatment with agonistic anti-JAML antibodies significantly reduced tumor volume in B16F10-OVA tumor-bearing wildtype (FIG. 7A), but not CD8^{-/-} mice (FIG. 7B), demonstrating that the observed effects are dependent on CD8⁺ T cells, but not on other JAML—expressing immune cell types (eg., myeloid subsets). Accordingly, while adoptive transfer of OVA antigen-specific JAML-sufficient OT-I CD8⁺ T cells (OT-I JAML^{wt}) decreased tumor growth, Crispr-Cas9-mediated depletion of JAML on transferred OT-I CD8⁺ T cells (OT-I JAML^{-/-}) reduced tumor control (FIG. 7C and FIG. 12A). Given that the frequencies of JAML-sufficient and JAML-deficient OT-I T cells in tumor tissues were comparable (FIG. 7D), it is unlikely that the observed differences in tumor control can be contributed to distinct migration tendencies or altered in vivo persistence. These data imply that CXADR (endogenous JAML ligand) might be expressed by cells in the TME, as JAML-expressing, but not JAML—deficient OT-I T cells, controlled tumor growth. Interestingly, Applicants found that B16F10 melanoma cells expressed CXADR, albeit at profoundly lower levels when compared to MC38 adenocarcinoma cells (FIG. 12B), implying that tumor cells might provide co-stimulation to JAML-expressing TILs. To test this hypothesis, Applicants utilized CRISPR-Cas9 to generate CXADR-deficient MC38-OVA cells (FIG. 12C) and performed an in vitro proliferation assay co-culturing either CXADR-sufficient or CXADR-deficient MC38-OVA tumor cells with CD8⁺ OT-I T cells. Notably, Applicants found significantly less proliferation of CD8⁺ OT-I T cells when they were co-cultured with CXADR^{-/-} MC38-OVA tumor cells (FIG. 12D), implying that tumor cells can provide co-stimulatory signals to CD8⁺ TILs via CXADR. As Applicants observed that agonistic anti-JAMIL antibody treatment decreased tumor growth in B16F10-OVA melanoma cells but not MC38-OVA tumor cells (FIG. 7A, 7E), Applicants reasoned that the disparate CXADR expression levels might be a critical determinant of anti-JAML treatment efficacy. To assess this hypothesis, Applicants inoculated mice with either CXADR^{+/+} or CXADR_{-/-} MC38-OVA cells and found that anti-JAML therapy was more effective in CXADR^{-/-} MC38-OVA treated mice (FIG. 7E), implying that tumor cells themselves trigger TIL activation via JAML. Utilizing TCGA data, Applicants verified the relatively lower expression of CXADR on melanoma samples when compared to other tumors of epithelial origins like esophageal, colonic and lung cancer (FIG. 12E), pointing to a possible immune evasion mechanism in melanoma and highlighting the potential benefit of agonistic anti-JAML antibody treatment specifically in human cancers expressing low levels of CXADR. Accordingly, CXADR expression on tumor cells might be utilized as an effective biomarker determining anti-JAML treatment efficacy.

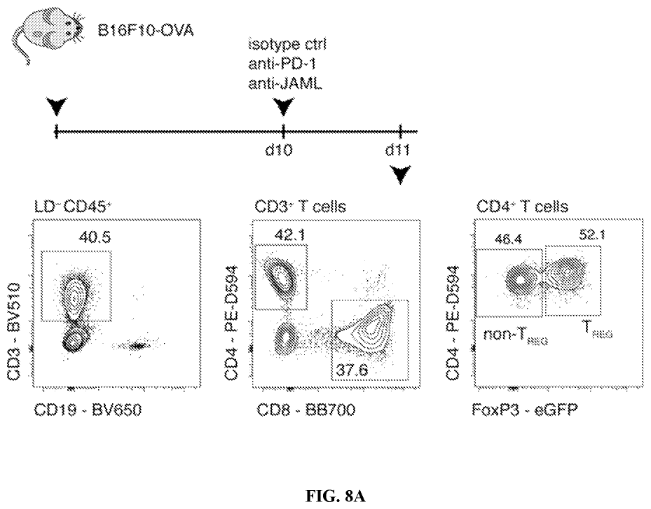

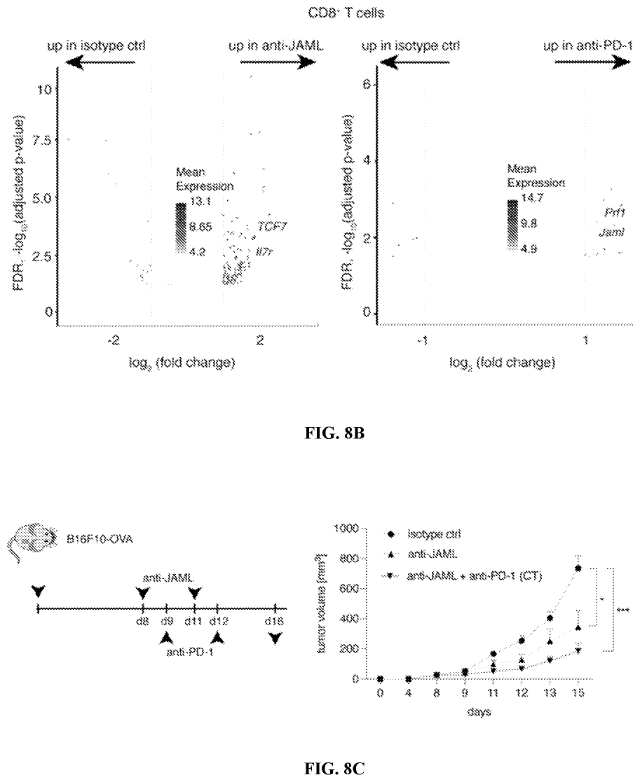

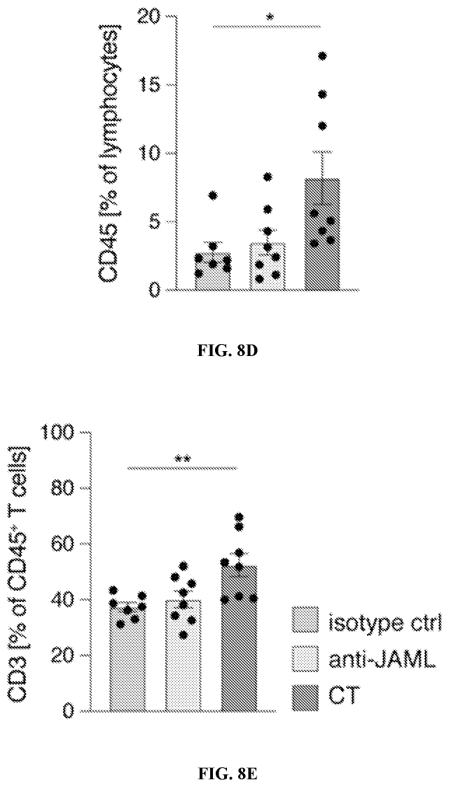

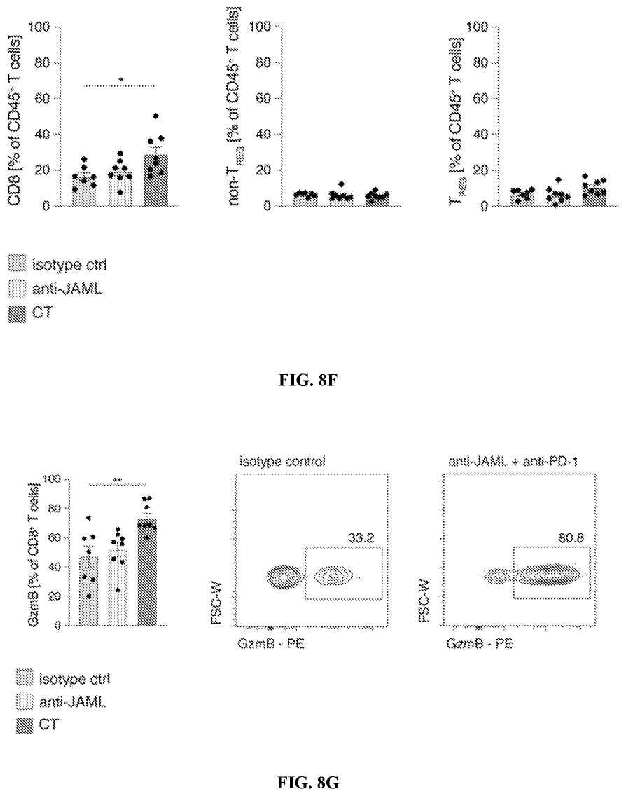

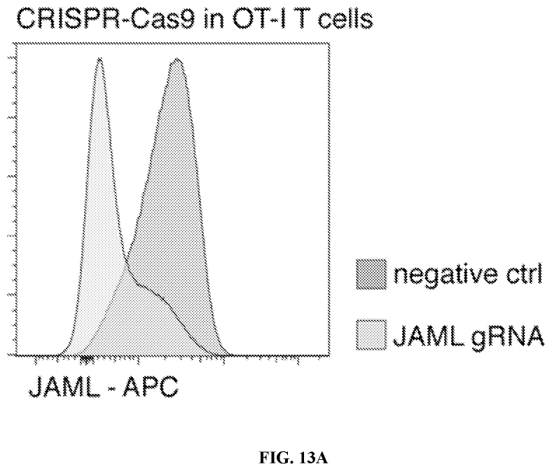

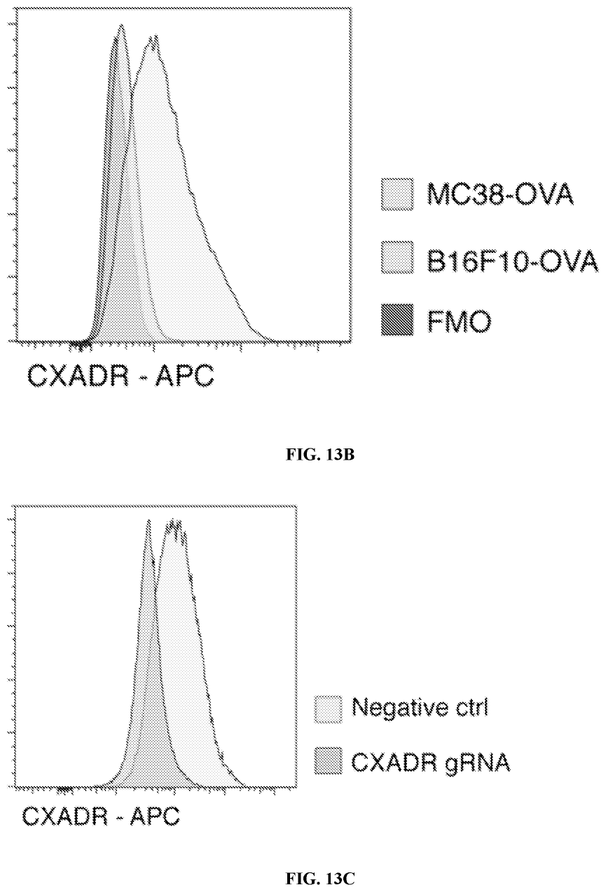

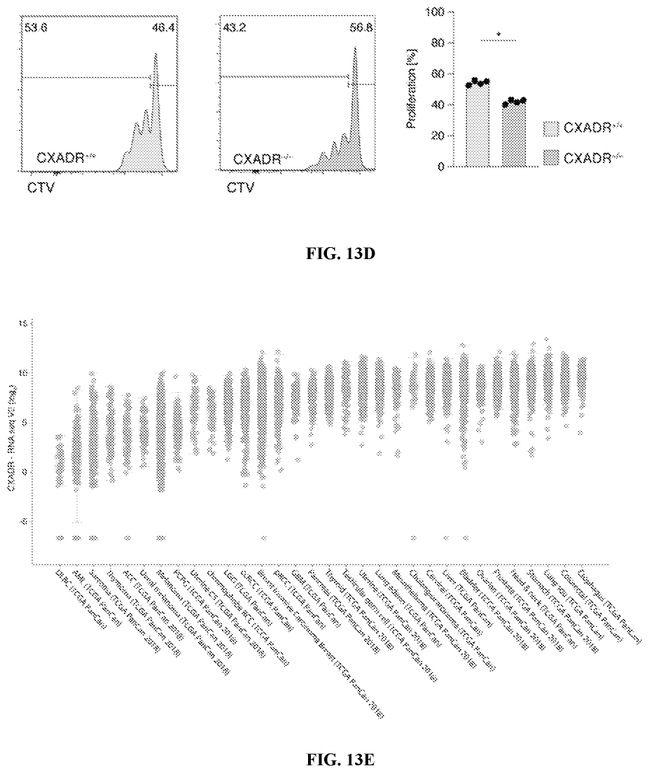

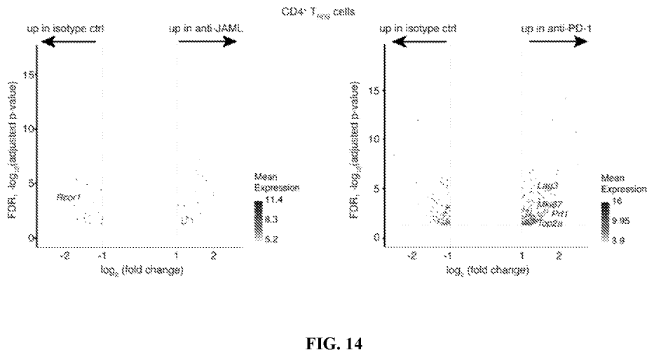

Synergistic anti-tumor effects in combination therapy with anti-PD-1 and anti-JAML. To elucidate molecular pathways selectively influenced by JAML signaling and to delineate the responsiveness of distinct TIL subsets to agonistic anti-JAML antibody and anti-PD1 treatment in vivo, Applicants performed RNA-seq analyses of sorted tumor-infiltrating CD4⁺ T_{REG} and CD8⁺ T cells (FIG. 8A). Pair-wise comparison of the bulk transcriptomes of tumor-infiltrating CD8⁺ T cells in treatment conditions versus isotype control showed that a greater number of genes were differentially expressed (DEG) in mice receiving agonistic anti-JAML therapy compared to anti-PD-1 therapy (151 versus 22 DEGs), and the converse was observed in tumor-infiltrating T_{REG} (45 versus 342 DEGs), (FIG. 8B and FIG. 13A). In line with Applicants' previous study⁷, Applicants found that anti-PD-1 therapy can activate suppressive T_{REG} cells, as evidenced by upregulation of several transcripts linked to functionality (Prf1, Lag3) and proliferation (Mki67, Top2a), while anti-JAML does not (FIG. 13A).

These findings confirmed that unlike anti-PD-1 therapies, agonistic anti-JAML antibodies preferentially target CD8⁺ TILs over immune suppressive T_{REG} cells due to its restricted expression profile (FIG. 8C). Importantly, anti-JAML treatment significantly increased the expression levels of genes (i.e., Tcf7, Il7r) shown to play a role in supporting ‘stem-like’ properties of T cells²⁶⁻²⁸, implying that anti-JAML therapy might either maintain or reinforce ‘stem-like’ phenotype in tumor-infiltrating CD8⁺ T cells (FIG. 8B). This result supports Applicants' hypothesis, generated from single-cell transcriptomic analysis of JAML-expressing CD8⁺ TILs (FIG. 8G-J), that ‘stem-like’ TILs are likely to be more responsive to agonistic anti-JAML antibodies when compared to anti-PD1 therapy.

Moreover, 2 of the most upregulated transcripts in CD8⁺ T cells by anti-PD-1 therapy were Prf1, encoding for Perforin, and interestingly, Jaml (FIG. 8B), indicative of an interconnected pathway between PD-1 signaling, which restricts TCR signaling and CD28 co-stimulation^{36,37}, and JAML expression, which Applicants found to be induced by TCR signaling. Thus, Applicants hypothesize that releasing TCR restriction with anti-PD-1 antibodies, upregulates the expression JAML on CD8⁺ T cells, which can then be targeted by agonistic anti-JAML antibodies, causing a further and selective activation of (tumor antigen-specific) CD8⁺ TILs. Based on these findings and given that anti-JAML agonistic antibody seems to reinforce a ‘stem-like’ phenotype and as multiple studies have identified T_{SCM} cells as pivotal mediators of anti-PD-1 treatment efficacy²⁶⁻²⁸, Applicants hypothesized that combination therapy (anti-JAML+anti-PD-1) is likely to result in improved tumor control. As before, Applicants utilized the B16F10-OVA model that is refractory to anti-PD-1 monotherapy. Importantly, Applicants found that anti-JAML/anti-PD-1 combination therapy resulted in greater reduction in tumor growth, demonstrating their synergistic effects (FIG. 8C). This enhanced anti-tumor response was associated with a significant increase in tumor-infiltrating lymphocytes (TILs) (FIG. 8D), and was predominantly mediated by elevated levels of CD8⁺ TILs, while the frequencies of CD4⁺ non-T_{REG} or CD4⁺ T_{REG} cells remained stable (FIG. 8 E,F). Notably, the proportion of granzyme B expressing CD8⁺ TILs was also significantly higher in combination therapy compared to anti-JAML monotherapy or isotype controls (FIG. 8G). Together, these findings provide mechanistic insights as to why agonistic anti-JAML therapy synergizes with anti-PD-1 treatment to improve tumor control.

### Experimental Discussion

Immunotherapies utilizing agonistic antibodies were initially considered to mainly activate the CD8⁺ T cell compartment, without appreciating potential effects on regulatory T cell subsets. However, subsequent studies have demonstrated that various immunotherapy drugs suffer from ‘on-target/off-cell effects’ and ‘on-target/off-tumor effects’, effectively dampening their treatment efficacy and clinical use. This initially underappreciated mechanism infers that T cell subsets other than CD8⁺ T cells (i.e. suppressive T_{REG} or T_{FR} cells) can express high levels of a given immunotherapy drug target in tumor tissues (on-target/off-cell effects). By binding and activating such suppressive cells, immunotherapies can create an immunosuppressive milieu and thus impede clinical potential and utility. Contrary to that, an overactivation of the immune system in normal tissues (on-target/off-tumor effects), frequently observed by non-specifically targeting T_{REG} cells with anti-CTLA-4 and further exacerbated by combination with anti-PD-1 therapy, can cause severe irAEs. Hence, there remains an unmet need for the development of immunotherapy targets that exhibit a more restricted expression profile.

Here Applicants show that the co-stimulatory molecule JAML is highly expressed in tumor-infiltrating CD8⁺ T_{RM} cells in multiple human cancer types, and that its expression is associated with enhanced functional potential of T_{RM} cells and also improved long-term survival outcomes in a cohort of HNSCC patients. Utilizing in vitro stimulation and CRISPR-Cas9 assays, Applicants found that JAML signaling through its endogenous ligand CXADR potently and selectively activates CD8⁺ T cells, and to a lesser degree, CD4⁺ T cells. These assays also revealed that JAML is potently induced by TCR signaling, implying that antigen-recognition drives JAML expression. Applicants demonstrated extensive 3D chromatin interactions between the promoters of CD3D and JAML in human T cells, but not other immune cell types like monocytes, implying that these cis-regulatory interactions might drive JAML expression. Crucially, these data suggest that agonistic anti-JAML antibodies might preferentially target and co-stimulate tumor-antigen specific CD8⁺ T cells, which upregulated JAML expression due to recent TCR engagement, in tumor tissues. Accordingly, anti-JAML therapy showed beneficial effects in a murine melanoma model, an effect that was dependent on CD8⁺ T cells. Moreover, Applicants found JAML expression to be predominantly restricted to CD8⁺ T cells in tumor tissue, with low expression in T cells from other organs or other T cell compartments, further substantiating the notion that it might specifically activate (antigen-specific) CD8⁺ T cells in the TME, thus reducing the risk of irAEs in non-malignant organs. Crucially, in murine tumors, Applicants found two distinct subsets of JAML-expressing CD8⁺ T cells; (i) a ‘stem-like’ population of CD8⁺ T cells expressing high levels of Tcf7, demonstrated to be pivotal for efficacious immune responses against viruses and tumors, and (ii), a Pdcd-1 enriched effector CTL cluster, likely driving anti-tumor effects. Furthermore, by uncovering an interconnected pathway between JAML and PD-1, Applicants' data provide mechanistic insights for the observed synergistic effects of anti-JAML and anti-PD-1 therapy, which significantly increased TIL infiltration and thus efficiently controlled tumor growth. While a recent study described JAML as a potential cancer immunotherapy target in mice¹⁸, Applicants' study provides critical insights into how anti-JAML agonistic antibody mediates its function and identify JAML as an immunotherapy target in tumor-infiltrating T_{RM} cells with a low risk of ‘off-cell’ and ‘off-tumor’ effects, features that are likely to enhance anti-tumor efficacy without causing significant irAEs in humans.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control. In some aspect, the publication is referenced by an Arabic numeral. The full citation for these publications are provided below.

### REFERENCES

1. Wolf, Y., Anderson, A. C. & Kuchroo, V. K. TIM3 comes of age as an inhibitory receptor. Nature Reviews Immunology vol. 20 173-185 (2020).
2. Manieri, N. A., Chiang, E. Y. & Grogan, J. L. TIGIT: A Key Inhibitor of the Cancer Immunity Cycle. Trends in Immunology vol. 38 20-28 (2017).
3. Wang, B. et al. Combination cancer immunotherapy targeting PD-1 and GITR can rescue CD8+ T cell dysfunction and maintain memory phenotype. Sci. Immunol. (2018) doi:10.1 126/sciimmunol.aat7061.
4. Gao, J. et al. VISTA is an inhibitory immune checkpoint that is increased after ipilimumab therapy in patients with prostate cancer. Nat. Med. 23, 551-555 (2017).
5. Andrews, L. P. et al. Resistance to PD1 blockade in the absence of metalloprotease-mediated LAG3 shedding. Sci. Immunol. 5(49):eabc2728 (2020).
6. Soldevilla, M. M. et al. ICOS Costimulation at the Tumor Site in Combination with CTLA-4 Blockade Therapy Elicits Strong Tumor Immunity. Mol. Ther. 27, 1878-1891 (2019).
7. Eschweiler, S. et al. Intratumoral follicular regulatory T cells curtail anti-PD-1 treatment efficacy. Nat. Immunol. 22(8):1052-1063 (2021)
8. Kumagai, S. et al. The PD-1 expression balance between effector and regulatory T cells predicts the clinical efficacy of PD-1 blockade therapies. Nat. Immunol. 21(11):1346-1358 (2020)
9. Ganesan, A.-P. et al. Tissue-resident memory features are linked to the magnitude of cytotoxic T cell responses in human lung cancer. Nat. Immunol. 18, 940-950 (2017).
10. Clarke, J. et al. Single-cell transcriptomic analysis of tissue-resident memory T cells in human lung cancer. J. Exp. Med. 216(9):2128-2149 (2019).
11. Savas, P. et al. Single-cell profiling of breast cancer T cells reveals a tissue-resident memory subset associated with improved prognosis. Nat. Med. 24, 986-993 (2018).
12. Okla, K., Farber, D. L. & Zou, W. Tissue-resident memory T cells in tumor immunity and immunotherapy. Journal of Experimental Medicine vol. 218(4):e20201605 (2021).
13. Simoni, Y. et al. Bystander CD8+ T cells are abundant and phenotypically distinct in human tumour infiltrates. Nat. 2018 5577706 557, 575-579 (2018).
14. Pardoll, D. M. The blockade of immune checkpoints in cancer immunotherapy. Nat. Rev. Cancer 12, 252-264 (2012).
15. Hellmann, M. D. et al. Nivolumab plus Ipilimumab in Lung Cancer with a High Tumor Mutational Burden. N. Engl. J. Med. 378(22):2093-2104 (2018)
16. Verdino, P., Witherden, D. A., Havran, W. L. & Wilson, I. A. The molecular interaction of CAR and JAML recruits the central cell signal transducer PI3K. Science 329(5996):1210-4 (2010)
17. Witherden, D. A. et al. The junctional adhesion molecule JAML is a costimulatory receptor for epithelial γδ T cell activation. Science 329(5996):1205-10. (2010)
18. McGraw, J. M. et al. JAML promotes CD8 and 76 T cell antitumor immunity and is a novel target for cancer immunotherapy. J. Exp. Med. 218(10):e20202644, (2021).
19. Buchan, S. L. et al. Antibodies to Costimulatory Receptor 4-1BB Enhance Anti-tumor Immunity via T Regulatory Cell Depletion and Promotion of CD8 T Cell Effector Function. Immunity 49(5):958-970 (2018)
20. Kamada, T. et al. PD-1+regulatory T cells amplified by PD-1 blockade promote hyperprogression of cancer. 116(20):9999-10008 (2019).
21. Edwards, J. et al. CD103+tumor-resident CD8⁺ T cells are associated with improved survival in immunotherapy-naïve melanoma patients and expand significantly during anti-PD-1 treatment. Clin. Cancer Res. 24, 3036-3045 (2018).
22. Park, S. L., Gebhardt, T. & Mackay, L. K. Tissue-Resident Memory T Cells in Cancer Immunosurveillance. Trends in Immunology vol. 40 735-747 (2019).
23. Park, S. L. et al. Tissue-resident memory CD8+ T cells promote melanoma-immune equilibrium in skin. Nature 565, 366-371 (2019).
24. Schmiedel, B. J. et al. Impact of Genetic Polymorphisms on Human Immune Cell Gene Expression. Cell 175, 1701-1715.e16 (2018).
25. Chandra, V. et al. Promoter-interacting expression quantitative trait loci are enriched for functional genetic variants. Nat. Genet. 53, 110-119 (2021).
26. Im, S. J. et al. Defining CD8+ T cells that provide the proliferative burst after PD-1 therapy. Nature 537, 417-421 (2016).
27. Siddiqui, I. et al. Intratumoral Tcf1+PD-1+CD8+ T Cells with Stem-like Properties Promote Tumor Control in Response to Vaccination and Checkpoint Blockade Immunotherapy. Immunity 50, 1-17 (2019).
28. Sade-Feldman, M. et al. Defining T Cell States Associated with Response to Checkpoint Immunotherapy in Melanoma. Cell 175, 998-1013.e20 (2018).
29. Scott, A. C. et al. TOX is a critical regulator of tumour-specific T cell differentiation. Nature 571(7764):270-274 (2019)
30. Bassez, A. et al. A single-cell map of intratumoral changes during anti-PD1 treatment of patients with breast cancer. Nat. Med. 275 27, 820-832 (2021).
31. Alfei, F. et al. TOX reinforces the phenotype and longevity of exhausted T cells in chronic viral infection. Nature 571(7764):265-269 (2019)
32. Wang, Z. et al. PD-1hi CD8+ resident memory T cells balance immunity and fibrotic sequelae. Sci. Immunol. 4(36):eaawl217 (2019).
33. Weber, D. A. et al. Neutrophil-derived JAML inhibits repair of intestinal epithelial injury during acute inflammation. Mucosal Immunol. 7, 1221-1232 (2014).
34. Alshetaiwi, H. et al. Defining the emergence of myeloid-derived suppressor cells in breast cancer using single-cell transcriptomics. Sci. Immunol. 5, 6017 (2020).
35. Kleffel, S. et al. Melanoma Cell-Intrinsic PD-1 Receptor Functions Promote Tumor Growth. Cell 162, 1242-1256 (2015).
36. Kamphorst, A. O. et al. Rescue of exhausted CD8 T cells by PD-1-targeted therapies is CD28-dependent. Science 355(6332):1423-1427 (2017).
37. Hui, E. et al. The T cell costimulatory receptor CD28 is a primary target of PD-1 mediated inhibition. Science 355(6332):1428-1433 (2016).
38. Almeida L G, Sakabe N J, deOliveira A R, Silva M C C, Mundstein A S, Cohen T, et al. CTdatabase: a knowledge-base of high-throughput and curated data on cancer-testis antigens. Nucleic Acids Res (2009) 37:D816-9. doi:10.1093/nar/gkn673.
39. Simpson A J G, Caballero O L, Jungbluth A, Chen Y-T, Old L J. Cancer/testis antigens, gametogenesis and cancer. Nat Rev Cancer (2005) 5:615-25. doi:10.1038/nrc1669.
40. Caballero O L, Chen Y-T. Cancer/testis (CT) antigens: potential targets for immunotherapy. Cancer Sci (2009) 100:2014-21. doi:10.1111/j.1349-7006. 2009.01303.x.
41. Bourre B. Targeting Tumor-Associated Antigens and Tumor-Specific Antigens. Crown Biosci. (2019) https://blog.crownbio.com/targeting-tumor-associated-antigens-and-tumor-specific-antigens.

1. A method of one or more of:
modulating an immune response to a tumor or cancer cell in a patient,
treating cancer or a tumor in a cancer patient; or
eliciting an anti-cancer or tumor response in a patient,
comprising modulating expression or activity of Junction Adhesion Molecule Like (JAML) in a T cell in the patient.

2. The method of claim 1, wherein the expression or activity of JAML is modulated by administering to the patient an effective amount of an agent that targets JAML in the T cell.

3. The method of claim 1, wherein the T cell is selected from the group of: an activated T cell in the subject; a tissue resident memory (TRM) cell; or a stem T cell.

4. The method of claim 3, wherein the activated T cell is specific for a tumor specific antigen or a tumor-associated antigen expressed by the tumor cell, wherein the tumor-associated antigen is optionally overexpressed by the tumor or cancer cell, the cancer or the tumor.

5. The method of claim 1, wherein the modulation comprises activating the T cell by agonizing the expression or activity of JAML in the T cell.

6. The method of claim 1, wherein the agent is bispecific and binds to JAML and binds to a second molecule expressed on the T cell.

7. The method of claim 6, wherein the second molecule is selected from a group of: CXCR5, CXCR6, CD8, CD103, CD49A, CD69, CD3, CD28, CD8 or PD1.

8. (canceled)

9. The method of claim 2, wherein the agent that binds to JAML comprises an agonistic antibody.

10. (canceled)

11. The method of claim 1, wherein the agent that targets JAML binds to a tumor antigen expressed by the tumor or cancer cell, tumor or cancer that is optionally overexpressed by the tumor cell, tumor or cancer.

12.-14. (canceled)

15. The method of claim 11, wherein the tumor antigen is selected from the group of: MAGE-D4B, PSMA, HER2, HER3, EGFR, AFP, CEA, CA-125, MUC-1, ETA, MUC-1, BAGE, GAGE-1, MAGE-A1, NY-ESO-1, Gp100, Melan-A/MART-1, Prostate-specific antigen, Mammoglobin-A, Alpha-fetoprotein, HER-2/neu, P53, K-ras, or TRP-2/INT2.

16.-22. (canceled)

23. A method for screening for a potential JAML anticancer therapy comprising contacting a first sample of T cells with an amount of a test agent that binds to JAML and a second agent that binds a tumor antigen, and assaying for increased expression of JAML in the T cell, wherein the test agent that increases expression of the JAML in the test cell is a potential JAML anticancer therapy.

24. (canceled)

25. (canceled)

26. A method of modulating JAML in a subject, comprising administering to the subject a bispecific antibody that targets and binds to JAML and a molecule expressed by a T cell.

27.-29. (canceled)

30. A method of diagnosing cancer, comprising contacting a sample isolated from the subject with an agent that detects the presence of JAML or CXADR in the sample isolated from the subject, wherein the presence of JAML or CXADR at higher or lower than baseline expression levels is diagnostic of cancer.

31. A method of diagnosing cancer in a subject comprising contacting T cells isolated from the subject or cancer sample isolated from the subject, with an antibody or agent that recognizes and binds to JAML, wherein the presence of an antibody-JAML complex in the sample is diagnostic of cancer.

32. A method of determining prognosis of a subject having cancer comprising measuring the density of CXADR expressing cells in a sample isolated from the subject, wherein a low density of cells indicates a more positive prognosis or wherein a high density of cells indicates a more negative prognosis, optionally wherein the more negative prognosis comprises a decreased probability in survival, and further optionally wherein the more positive prognosis comprises an increased probability in survival.

33. A method of determining prognosis of a subject having cancer comprising contacting T cells isolated from the subject with an antibody or agent that recognizes and binds to JAML to determine the frequency of T cells expressing JAML in tumor cells, wherein a high density of JAML in T cells indicates a more positive prognosis or wherein a low density of JAML in T cells indicates a more negative prognosis, optionally wherein the more negative prognosis comprises a decreased probability in survival, and further optionally wherein the more positive prognosis comprises an increased probability in survival.

34. A method of determining the responsiveness of a subject to cancer therapy comprising contacting T cells isolated from the subject with an antibody or agent that recognizes and binds to JAML and detecting any antibody-JAML or agent-JAML complex to determine the frequency of JAML expressing T cells in the subject, wherein a high frequency of JAML T cells indicates an increased likelihood of responsiveness to a cancer therapy.

35. A method of identifying a subject that is likely to respond to a cancer therapy, comprising contacting a sample isolated from the subject with an agent that detects the presence of CXADR in the sample and detecting any agent-CXADR complex, wherein the presence of CXADR at lower than baseline expression levels indicates that the subject is likely to respond to the cancer therapy.

36.-39. (canceled)

40. The method of claim 30, further comprising administering a cancer therapy to the subject.

41. The method of claim 40, wherein the cancer therapy is an agent that binds to JAML.

42.-52. (canceled)


SEQUENCE LISTING (9 sequences)

SEQ ID NO: 1 - protein, 6 aa, synthetic construct
HHHHHH

SEQ ID NO: 2 - RNA, 250 nt, synthetic construct
  source 1..250: mol_type = other RNA
  misc_difference 1..250: note = This sequence may encompass 100-250 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - protein, 274 aa, Homo sapiens
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCAPVLSGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 4 - protein, 95 aa, Homo sapiens
DSAVYLCASSLLRVYEQYFGPGTRLTVTEDLKNVFPPEVAVFEPPEAEISHTQKATLVCLATGFYPDHVELSWWVNGKEVHSGVSTDPQPLKEQP

SEQ ID NO: 5 - protein, 394 aa, Homo sapiens
MFCPLKLILLPVLLDYSLGLNDLNVSPPELTVHVGDSALMGCVFQSTEDKCIFKIDWTLSPGEHAKDEYVLYYYSNLSVPIGRFQNRVHLMGDILCNDGSLLLQDVQEADQGTYICEIRLKGESQVFKKAVVLHVLPEEPKELMVHVGGLIQMGCVFQSTEVKHVTKVEWIFSGRRAKEEIVFRYYHKLRMSVEYSQSWGHFQNRVNLVGDIFRNDGSIMLQGVRESDGGNYTCSIHLGNLVFKKTIVLHVSPEEPRTLVTPAALRPLVLGGNQLVIIVGIVCATILLLPVLILIVKKTCGNKSSVNSTVLVKNTKKTNPEIKEKPCHFERCEGEKHIYSPIIVREVIEEEEPSEKSEATYMTMHPVWPSLRSDRNNSLEKKSGGGMPKTQQAF

SEQ ID NO: 6 - DNA, 40 bp, Homo sapiens
  source 1..40: mol_type = genomic DNA
gtcaccaggagagcacgccaaggtaacgcgggggaaacat

SEQ ID NO: 7 - DNA, 17 bp, synthetic construct
  source 1..17: mol_type = other DNA
gtcaccaggagagcacg

SEQ ID NO: 8 - DNA, 41 bp, Homo sapiens
  source 1..41: mol_type = genomic DNA
gtcaccaggagagcacgggggaaaaacaggggggaaaaaaa

SEQ ID NO: 9 - protein, 4 aa, synthetic construct
NANP